(19) United States
(12) Patent Application Publication
(10) Pub. No.: US 2003/0236308 A1
(43) Pub. Date: Dec. 25, 2003

(54) Compositions of cyclooxygenase-2 selective inhibitors and acetaminophen for treatment and prevention of inflammation, inflammation-mediated disorders and pain

(75) Inventor: Karen Seibert, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/246,846

(22) Filed: Sep. 18, 2002

Related U.S. Application Data
(60) Provisional application No. 60/322,980, filed on Sep. 18, 2001.

(51) Int. Cl. A61K 31/195

(52) U.S. Cl. 514/567

(57) ABSTRACT

A composition is provided comprising a cyclooxygenase-2 selective inhibitor and acetaminophen. The composition is effective for the treatment and prevention of inflammation, an inflammation-mediated disorder, and pain.

### CROSS REFERENCE TO RELATED APPLICATION

This application claims priority from Provisional Application Serial No. 60/322,980 filed on Sep. 18, 2001, which is hereby incorporated by reference in its entirety.

### FIELD OF THE INVENTION

The present invention provides a composition for the treatment and prevention of inflammation, inflammation-mediated disorders and pain. More particularly, the invention is directed toward a composition of a cyclooxygenase-2 selective inhibitor and acetaminophen that is effective in the treatment and prevention of inflammation, inflammation-mediated disorders and pain.

### BACKGROUND OF THE INVENTION

Inflammatory disease is any disease marked by inflammation, which is a localized protective response elicited by injury or destruction of tissues and serves to destroy, dilute, or wall off both the injurious agent and the injured tissue. The classical signs of pain, heat, redness, swelling and loss of function characterize inflammation in the acute form. Inflammation occurs when, upon injury, recruited polymorphonuclear leukocytes release reactive oxygen species (ROS) in oxidative bursts resulting in a complex cascade of events. Histologically, it involves a complex series of events, including dilation of arterioles, capillaries, and venules, with increased permeability and blood flow; exudation of fluids, including plasma proteins; and leukocytic migration into the inflammatory focus.

The inflammation of the inflammatory diseases may be caused by a multitude of mediators including radiant, mechanical, chemical, infectious, and immunological stimuli. One such mediator of the inflammatory response is the prostaglandins. Prostaglandins are a potent class of biologically active lipid derivatives that mediate the inflammatory response by inhibiting platelet aggregation, increasing vascular permeability, increasing vascular dilation, inducing smooth-muscle contraction and causing the induction of neutrophil chemotaxis.

The biosynthesis of prostaglandins from arachidonic acid occurs in a three step process that includes 1) hydrolysis of arachidonic acid from phospholipid precursors catalyzed by a phospholipase A₂; 2) cyclooxygenase (“COX”) catalyzed oxygenation of arachidonic acid to prostaglandin G2 (“PGG2”); and 3) conversion of prostaglandin G2 to the biologically active end product, prostaglandin, catalyzed by a series of synthases and reductases. Of these three steps, the COX catalyzed reaction is the first committed and rate limiting step in prostaglandin synthesis.

Because of its key role in prostaglandin biosynthesis, COX enzyme inhibition is a natural target as a means to inhibit prostaglandin production. It is now known that two gene products possessing COX enzyme activity are expressed, termed COX-1 and COX-2. COX-1 was the first discovered isoform and is constitutively expressed in most tissue types. Because it is constitutively expressed, COX-1 participates in several “house-keeping” functions including the regulation of vascular homeostasis, maintenance of gastrointestinal integrity, and maintenance of kidney function. COX-2, on the other hand, is inducibly expressed in response to numerous inflammatory-related stimuli, and is, therefore, believed to be responsible for mediating the production of prostaglandins that participate in the inflammatory response and inflammatory related disorders.

Nonsteroidal anti-inflammatory agents (NSAIDs), such as aspirin or ibuprofen, provide one means to reduce effects associated with the prostaglandin mediated inflammatory response. These potent anti-inflammatory agents exert their effect by non-selectively inhibiting COX-1 and COX-2 activity. Inhibition of prostaglandin synthesis by NSAIDs is anti-pyretic, analgesic, anti-inflammatory, and anti-thrombogenic. Because NSAID's non-selectively inhibit both COX-1 and COX-2, however, they are active not only in reducing the prostaglandin-induced pain and swelling associated with the inflammation process (i.e. COX-2 mediated), but are also active in affecting other prostaglandin-regulated processes not associated with the inflammation process (COX-1 mediated). As a result, the use of high doses of most common NSAIDs can produce severe side effects, including life-threatening ulcers that limit their therapeutic potential.

Acetaminophen is also utilized as a means to reduce effects associated with the inflammatory response. In fact it, is now the most popular over-the-counter drug for inflammatory related symptoms. Acetaminophen has potent analgesic and antipyretic actions. Because of these properties, it is generally regarded as an NSAID. Acetaminophen, however, unlike other NSAIDs, has very little anti-inflammatory activity. In addition, it lacks other typical actions of NSAIDS, such as antiplatelet activity and gastrotoxicity. Despite its wide spread use, however, the mechanism of action of acetaminophen has yet to be satisfactorily characterized. It only weakly inhibits COX-1 and COX-2 in vitro, but markedly reduces prostaglandin synthesis in vivo. Because of this low sensitivity of both COX-1 and COX-2 to acetaminophen, it has been postulated that a third COX enzyme may exist which can be inhibited by acetaminophen (Vane et al., (1998) Annu. Rev.Pharmacol Toxicol.38: 97-120).

Another widely employed means to inhibit prostaglandin-mediated inflammation is through the use of COX-2 selective inhibitors. These COX-2 selective inhibitors have been shown to be anti-inflammatory without the associated gastric and kidney related toxicity problems. Several drugs that are COX-2 selective inhibitors have been developed. For example, compounds that selectively inhibit COX-2 have been described in U.S. Pat. Nos. 5,380,738; 5,344,991; 5,393,790; 5,434,178; 5,474,995; 5, 510,368 and WO documents WO96/06840, WO96/03388, WO96/03387, WO96/19469, WO96/25405, WO95/15316, WO94/15932, WO94/27980, WO95/00501, WO94/13635, WO94/20480, and WO94/26731. It has been reported, however, that COX-2 selective inhibitors are not as active in tests that measure acute pain states (Bannwarth, B. (2001) Arch. Intern. Med.161: 127) as would be desired.

Accordingly, a need exists for the prevention and treatment of inflammation, inflammatory-mediated disorders, and pain that is analgesic, anti-pyretic, anti-inflammatory, and anti-thrombogenic, but without the gastrotoxicity and kidney problems associated with conventional NSAIDS.

### SUMMARY OF THE INVENTION

Among the several aspects of the invention is provided a method and a composition for the treatment or prevention of inflammation, an inflammation-mediated disorder, or pain in a subject. The composition comprises a cyclooxygenase-2 selective inhibitor or a pharmaceutically acceptable salt or prodrug thereof and acetaminophen or a pharmaceutically acceptable salt or prodrug thereof. Further, the method comprises administering to the subject a cyclooxygenase-2 selective inhibitor or a pharmaceutically acceptable salt or prodrug thereof and acetaminophen or a pharmaceutically acceptable salt or prodrug thereof.

In one embodiment, the cyclooxygenase-2 selective inhibitor is a phenyl acetic acid derivative.

In still another embodiment, the cyclooxygenase-2 selective inhibitor is a compound of the formula or pharmaceutically acceptable salt, ester, or prodrug thereof:

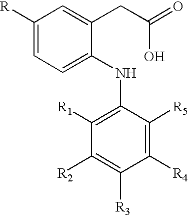

Wherein:

R₁ is methyl or ethyl;

R₁ is chloro or fluoro;

R₂ is hydrogen or fluoro

R₃ is hydrogen, fluoro, chloro, methyl, ethyl, methoxy, ethoxy or hydroxy;

R₄ is hydrogen or fluoro; and

R₅ is chloro, fluoro, trifluoromethyl or methyl, provided that R₁, R₂, R₄ and R₅ are not all fluoro when R is ethyl and R₃ is H.

Abbreviations and Definitions

To facilitate understanding of the invention, a number of terms and abbreviations as used herein are defined below:

The terms “cyclooxygenase-2” and “COX-2” are used interchangeably herein.

The term “prevention” includes either preventing the onset of clinically evident inflammation or an inflammation mediated disorder or pain altogether or preventing the onset of a preclinically evident stage of inflammation or an inflammation mediated disorder or pain in individuals. This definition includes prophylactic treatment.

The term “inhibition” as used herein means to decrease the severity of inflammation or an inflammation mediated order or pain as compared to that which would occur in the absence of the application of the method of the present invention.

The phrase “therapeutically-effective” is intended to qualify the amount of each agent which will achieve the goal of improvement in disorder severity and the frequency of incidence over no treatment or treatment of each agent by itself, while avoiding adverse side effects typically associated with alternative therapies.

The term “subject” for purposes of treatment includes any human or animal subject who is susceptible to inflammation, an inflammation-mediated disorder or pain. The subject can be a domestic livestock species, a laboratory animal species, a zoo animal or a companion animal. In one embodiment, the subject is a human being.

The term “COX-2 selective inhibitor” denotes a compound able to inhibit COX-2 without significant inhibition of cyclooxygenase-1. Preferably, it includes compounds that have a COX-2 IC₅₀ of less than about 0.2 micro molar, and also have a selectivity ratio of COX-2 inhibition over cyclooxygenase-1 inhibition of at least 50, and more preferably of at least 100. Even more preferably, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl,” “lower alkenyl,” embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl”, radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “halo” means halogens such as fluorine, chlorine, bromine or iodine.

The term “haloalkyl” embraces radicals wherein any one or more of the alkyl carbon atoms is substituted with halo defined as above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals.

“Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl. The term “sulfonyl”, whether used alone or linked to other terms such as alkylsulfonyl, denotes respectively divalent radicals —SO₂—.

“Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl,” “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “carbonyl”, whether used alone or with other terms, such as “alkoxycarbonyl”, denotes —(C═O)—.

The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H.

The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl.

The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl,” “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aralkoxy” embraces aralkyl radicals attached through an oxygen atom to other radicals.

The term “aralkoxyalkyl” embraces aralkoxy radicals attached through an oxygen atom to an alkyl radical.

The term “aralkylthio” embraces aralkyl radicals attached to a sulfur atom.

The term “aralkylthioalkyl” embraces aralkylthio radicals attached through a sulfur atom to an alkyl radical. The term “aminoalkyl” embraces alkyl radicals substituted with one or more amino radicals. More preferred are “lower aminoalkyl” radicals. Examples of such radicals include aminomethyl, aminoethyl, and the like.

The term “alkylamino” denotes amino groups that have been substituted with one or two alkyl radicals. Preferred is “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “arylamino” denotes amino groups that have been substituted with one or two aryl radicals, such as N-phenylamino.

The “arylamino” radicals may be further substituted on the aryl ring portion of the radical.

The term “aralkylamino” embraces aralkyl radicals attached through an amino nitrogen atom to other radicals.

The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl. The term “aminocarbonyl” denotes an amide group of the formula —C(═O)NH₂.

The term “alkylaminocarbonyl” denotes an aminocarbonyl group that has been substituted with one or two alkyl radicals on the amino nitrogen atom. Preferred is “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The term “alkylaminoalkyl”, embraces radicals having one or more alkyl radicals attached to an aminoalkyl radical.

The term “aryloxyalkyl” embraces radicals having an aryl radical attached to an alkyl radical through a divalent oxygen atom.

The term “arylthioalkyl” embraces radicals having an aryl radical attached to an alkyl radical through a divalent sulfur atom.

The compounds utilized in the current invention may be present in the form of free bases or pharmaceutically acceptable acid addition salts thereof. The term “pharmaceutically-acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts of compounds include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine.

### DESCRIPTION OF THE PREFERRED EMBODIMENTS

It has been discovered that treatment or prevention of inflammation, inflammation mediated disorders, and pain is provided by a combination therapy comprising a therapeutically effective amount of a selective COX-2 inhibitor along with a therapeutically effective amount of acetaminophen. This combination therapy is beneficial, without being held to any particular theory, because it is believed that the selective COX-2 inhibitor and acetaminophen each attenuate inflammation and pain via an independent mechanism. Thus, the coupling of a COX-2 selective inhibitor and acetaminophen provides a synergistic therapy for the treatment and prevention of inflammation, inflammation-mediated disorders, and pain. The use of COX-2 selective inhibitors is also highly advantageous in the present co-therapy because it minimizes the gastric side effects that can occur with non-selective NSAIDs, especially where prolonged treatment is expected.

Generally speaking, any cyclooxygenase-2 selective inhibitor or prodrug or pharmaceutically acceptable salt thereof may be employed in the method of the present invention. In one embodiment, the cyclooxygenase-2 selective inhibitor can be, for example, the cyclooxygenase-2 selective inhibitor meloxicam, Formula B-1 (CAS registry number 71125-38-7) or a pharmaceutically acceptable salt or prodrug thereof.

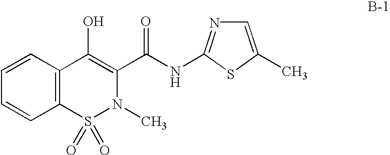

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or a pharmaceutically acceptable salt or prodrug thereof.

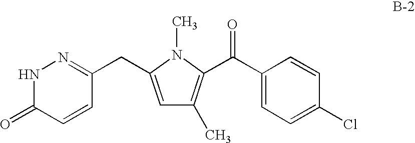

In a preferred embodiment the cyclooxygenase-2 selective inhibitor is preferably of the chromene structural class that is a substituted benzopyran or a substituted benzopyran analog, and even more preferably selected from the group consisting of substituted benzothiopyrans, dihydroquinolines, or dihydronaphthalenes having the general Formula I shown below and possessing, by way of example and not limitation, the structures disclosed in Table 1, including the diastereomers, enantiomers, racemates, tautomers, salts, esters, amides and prodrugs thereof. Furthermore, benzopyran cyclooxygenase-2 selective inhibitors useful in the practice of the present methods are described in U.S. Pat. No. 6,034,256 and 6,077,850 herein incorporated by reference in their entirety.

In one embodiment, the cyclooxygenase-2 selective inhibitor is of the chromene structural class and is represented by Formula I or an isomer, a pharmaceutically acceptable salt, ester, or prodrug thereof:

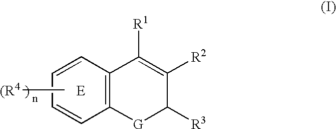

wherein n is an integer which is 0, 1, 2, 3 or 4;

wherein G is O, S or NR^{a};

wherein R^{a} is alkyl;

wherein R1 is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

G is O, S or NR^{b};

R¹ is H;

R^{b} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or an isomer, a pharmaceutically acceptable salt, ester, or prodrug thereof; wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof: wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

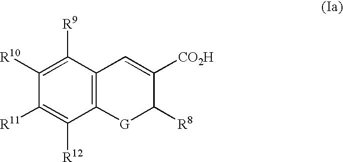

or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE 1
Examples of Chromene Cyclooxygenase-2
Selective Inhibitors as Embodiments

| Compound | |
|---|---|
| Number | Structural Formula |
| B-3 | 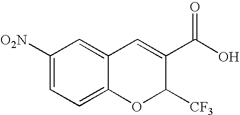 |
| | 6-Nitro-2-trifluoromethyl-2H-1- |
| | benzopyran-3-carboxylic acid |
| B-4 | 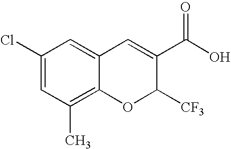 |
| | 6-Chloro-8-methyl-2-trifluoromethyl- |
| | 2H-1-benzopyran-3-carboxylic acid |
| B-5 | 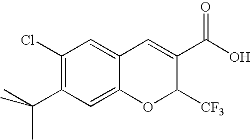 |
| | ((S)-6-Chloro-7-(1,1-dimethylethyl)-2- |
| | (trifluoromethyl-2H-1-benzopyran-3-carbaxylic |
| | acid |
| B-6 | 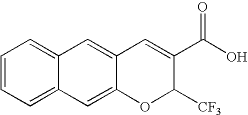 |
| | 2-Trifluoromethyl-2H-naphtho[2,3-b] |
| | pyran-3-carboxylic acid |
| B-7 | 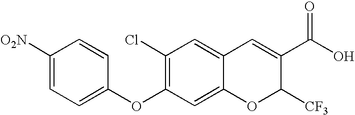 |
| | 6-Chloro-7-(4-nitrophenoxy)-2-(trifluoromethyl)- |
| | 2H-1-benzopyran-3-carboxylic acid |
| B-8 | 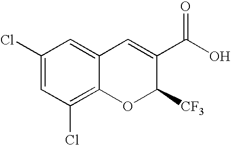 |
| | ((S)-6,8-Dichloro-2-(trifluoromethyl)- |
| | 2H-1-benzopyran-3-carboxylic acid |
| B-9 | 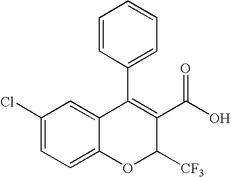 |
| | 6-Chloro-2-(trifluoromethyl)-4-phenyl- |
| | 2H-1-benzopyran-3-carboxylic acid |
| B-10 | 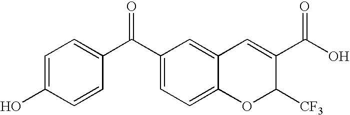 |
| | 6-(4-Hydroxybenzoyl)-2-(trifluoromethyl)- |
| | 2H-1-benzopyran-3-carboxylic acid |
| B-11 | 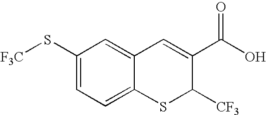 |
| | (Trifluoromethyl)-6-[(trifluoromethyl)thio]- |
| | 2H-1-benzothiopyran-3-carboxylic acid |
| B-12 | 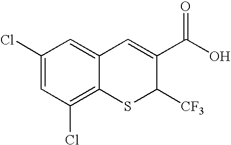 |
| | 6,8-Dichloro-2-trifluoromethyl-2H-1- |
| | benzothiopyran-3-carboxylic acid |
| B-13 | 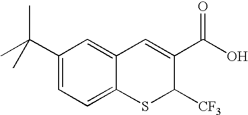 |
| | 6-(1,1-Dimethylethyl)-2-(trifluoromethyl)- |
| | 2H-1-benzothiopyran-3-carboxylic acid |
| B-14 | 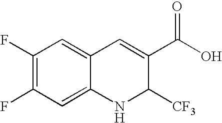 |
| | 6,7-Difluoro-1,2-dihydro-2-(trifluoro |
| | methyl)-3-quinolinecarboxylic acid |
| B-15 | 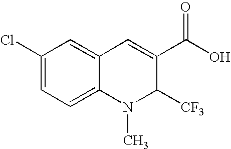 |
| | 6-Chloro-1,2-dihydro-1-methyl-2-(trifluoro |
| | methyl)-3-quinolinecarboxylic acid |
| B-16 | 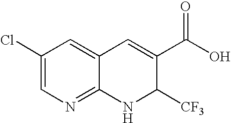 |
| | 6-Chloro-2-(trifluoromethyl)-1,2-dihydro |
| | [1,8]naphthyridine-3-carboxylic acid |
| B-17 | 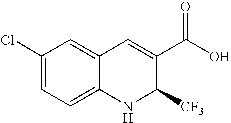 |
| | ((S)-6-Chloro-1,2-dihydro-2-(trifluoro |
| | methyl)-3-quinolinecarboxylic acid |

In a further preferred embodiment, the cyclooxygenase inhibitor is selected from the class of tricyclic cyclooxygenase-2 selective inhibitors represented by the general structure of Formula II:

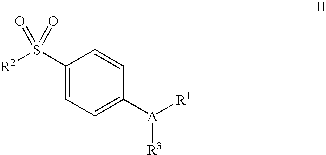

wherein A is selected from the group consisting of partially unsaturated or unsaturated heterocyclyl and partially unsaturated or unsaturated carbocyclic rings;

wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is selected from the group consisting of methyl or amino; and

wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

In a still more preferred embodiment of the invention the cyclooxygenase-2 selective inhibitor represented by the above Formula II is selected from the group of compounds, illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), JTE-522 (B-23), or an isomer, ester, a pharmaceutically acceptable salt or prodrug thereof.

TABLE 2
Examples of Tricyclic Cyclooxygenase-2
Selective Inhibitors as Embodiments

| Compound | |
|---|---|
| Number | Structural Formula |
| B-18 | 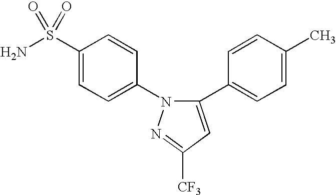 |
| B-19 | 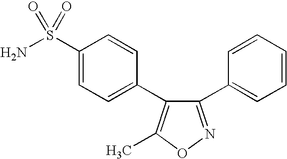 |
| B-20 | 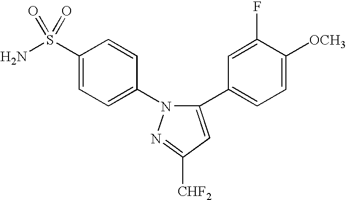 |
| B-21 | 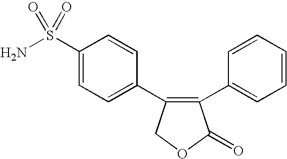 |
| B-22 | 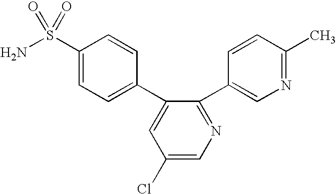 |
| B-23 | 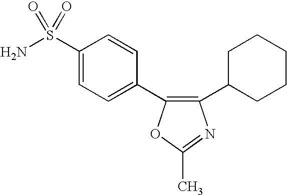 |

In an even more preferred embodiment, the cyclooxygenase-2 selective inhibitor is selected from the group consisting of celecoxib, rofecoxib and etoricoxib.

In another highly preferred embodiment of the invention, parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

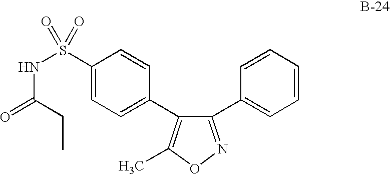

A preferred form of parecoxib is sodium parecoxib.

In another preferred embodiment of the invention, the compound having the formula B-25 that has been previously described in International Publication number WO 00/24719 (which is herein incorporated by reference), is another tricyclic cyclooxygenase-2 selective inhibitor which may be advantageously employed.

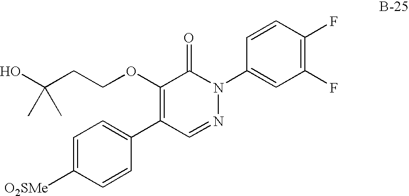

Another preferred cyclooxygenase-2 selective inhibitor that is useful in connection with the method(s) of the present invention is N-(2-cyclohexyloxynitrophenyl)-methane sulfonamide (NS-398) having a structure shown below as B-26.

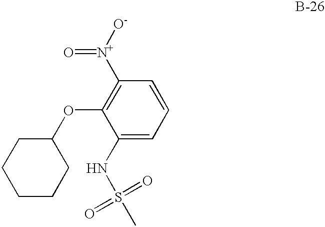

In yet a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof:

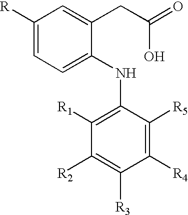

Wherein:

R is methyl or ethyl;

R₁ is chloro or fluoro;

R₂ is hydrogen or fluoro

R₃ is hydrogen, fluoro, chloro, methyl, ethyl, methoxy, ethoxy or hydroxy;

R₄ is hydrogen or fluoro; and

R₅ is chloro, fluoro, trifluoromethyl or methyl,

provided that R₁, R₂, R₄ and R₅ are not all fluoro when R is ethyl and R₃ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:

R is ethyl;

R¹ and R² are chloro;

R³ and R⁴ are hydrogen; and

and R⁵ is methyl.

In yet another embodiment, the cyclooxygenase-2 selective inhibitor is represented by Formula (IV):

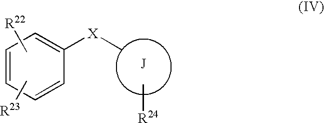

or an isomer, a pharmaceutically acceptable salt, an ester, or a prodrug thereof,

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V):

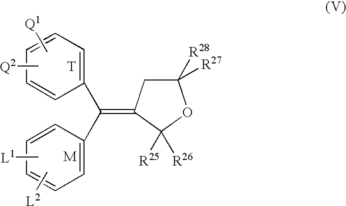

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In a particularly preferred embodiment, the compounds N-(2-cyclohexyloxynitrophenyl)methane sulfonamide, and (E)-4-[(4-methylphenyl)(tetrahydro-2-oxo-3-furanylidene) methyl] benzenesulfonamide having the structure of Formula (V) are employed as cyclooxygenase-2 selective inhibitors.

Exemplary compounds that are useful for the cyclooxygenase-2 selective inhibitor in connection with the method(s) of the present invention, the structures for which are set forth in Table 3 below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231);

S-2474 (B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid;

or an isomer, a pharmaceutically acceptable salt, ester or prodrug thereof.

TABLE 3
Examples of Cyclooxygenase-2 Selective
Inhibitors as Embodiments

| Compound | |
|---|---|
| Number | Structural Formula |
| B-26 | 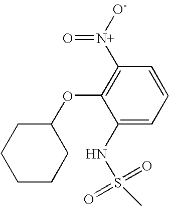 |
| | N-(2-cyclohexyloxynitrophenyl) methane sulfonamide or NS-398; |
| B-27 | 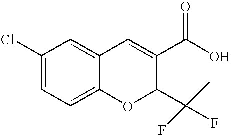 |
| | 6-chloro-2-trifluoromethyl-2H-1-benzopyran-3-carboxylic acid; |
| B-28 | 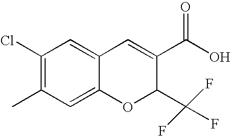 |
| | 6-chloro-7-methyl-2-trifluoromethyl-2H-1-benzopyran-3-carboxylic acid; |
| B-29 | 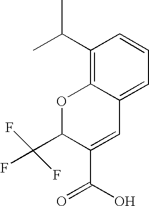 |
| | 8-(1-methylethyl)-2-trifluoromethyl-2H-1-benzopyran-3-carboxylic acid; |
| B-30 | 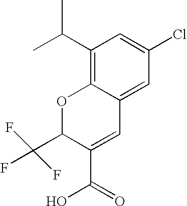 |
| | 6-chloro-8-(1-methylethyl)-2-trifluoromethyl- |
| | 2H-1-benzopyran-3-carboxylic acid; |
| B-31 | 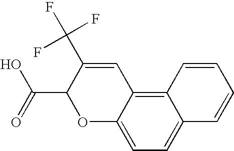 |
| | 2-trifluoromethyl-3H-naphtho[2,1-b]pyran-3-carboxylic acid; |
| B-32 | 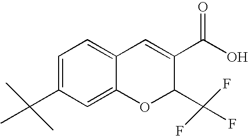 |
| | 7-(1,1-dimethylethyl)-2-trifluoromethyl-2H-1-benzopyran- |
| | 3-carboxylic acid; |
| B-33 | 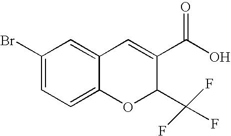 |
| | 6-bromo-2-trifluoromethyl-2H-1-benzopyran-3-carboxylic acid; |
| B-34 | 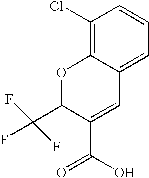 |
| | 8-chloro-2-trifluoromethyl-2H-1-benzopyran-3-carboxylic acid; |
| B-35 | 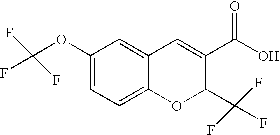 |
| | 6-trifluoromethoxy-2-trifluoromethyl-2H-1-benzopyran-3-carboxylic acid; |
| B-36 | 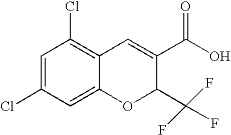 |
| | 5,7-dichloro-2-trifluoromethyl-2H-1-benzopyran-3-carboxylic acid; |
| B-37 | 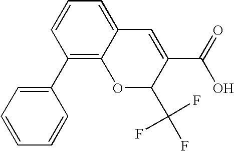 |
| | 8-phenyl-2-trifluoromethyl-2H-1-benzopyran-3-carboxylic acid; |
| B-38 | 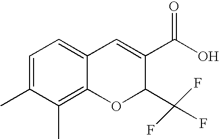 |
| | 7,8-dimethyl-2-trifluoromethyl-2H-1-benzopyran-3-carboxylic acid; |
| B-39 | 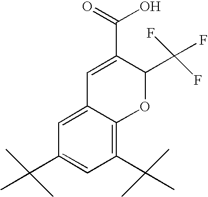 |
| | 6,8-bis(dimethylethyl)-2-trifluoromethyl-2H-1-benzopyran- |
| | 3-carboxylic acid; |
| B-40 | 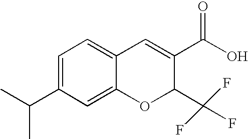 |
| | 7-(1-methylethyl)-2-trifluoromethyl-2H-1-benzopyran-3-carboxylic acid; |
| B-41 | 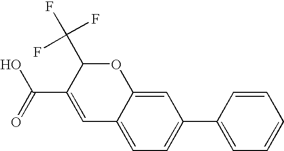 |
| | 7-phenyl-2-trifluoromethyl-2H-1-benzopyran-3-carboxylic acid; |
| B-42 | 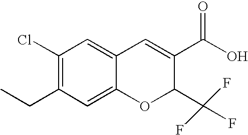 |
| | 6-chloro-7-ethyl-2-trifluoromethyl-2H-1-benzopyran-3-carboxylic acid; |
| B-43 | 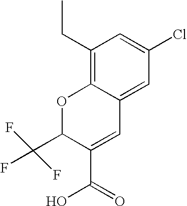 |
| | 6-chloro-8-ethyl-2-trifluoromethyl-2H-1-bcnzopyran-3-carboxylic acid; |
| B-44 | 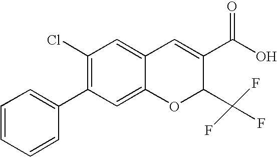 |
| | 6-chloro-7-phenyl-2-trifluoromethyl-2H-1-benzopyran-3-carboxylic acid; |
| B-45 | 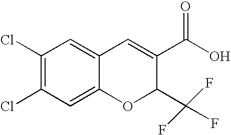 |
| | 6,7-dichloro-2-trifluoromethyl-2H-1-benzopyran-3-carboxylic acid; |
| B-46 | 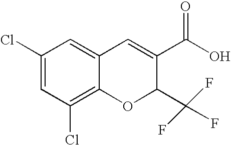 |
| | 6,8-dichloro-2-trifluoromethyl-2H-1-benzopyran-3-carboxylic acid; |
| B-47 | 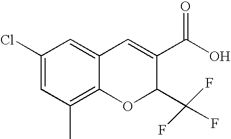 |
| | 6-chloro-8-methyl-2-trifluoromethyl-2H-1-benzopyran-3-carboxylic acid; |
| B-48 | 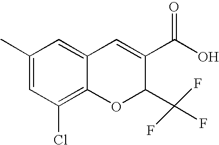 |
| | 8-chloro-6-methyl-2-trifluoromethyl-2H-1-benzopyran-3-carboxylic acid; |
| B-49 | 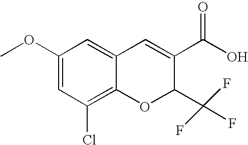 |
| | 8-chloro-6-methoxy-2-trifluoromethyl-2H-1-benzopyran-3-carboxylic acid; |
| B-50 | 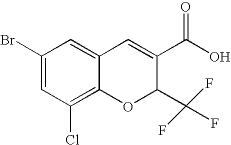 |
| | 6-bromo-8-chloro-2-trifluoromethyl-2H-1-benzopyran-3-carboxylic acid; |
| B-51 | 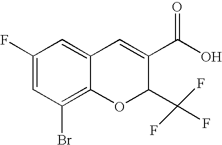 |
| | 8-bromo-6-fluoro-2-trifluoromethyl-2H-1-benzopyran-3-carboxylic acid; |
| B-52 | 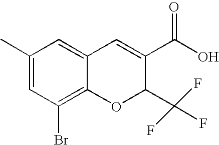 |
| | 8-bromo-6-methyl-2-trifluoromethyl-2H-1-benzopyran-3-carboxylic acid; |
| 2-53 | 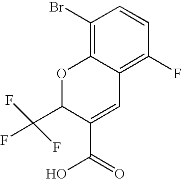 |
| | 8-bromo-5-fluoro-2-trifluoromethyl-2H-1-benzopyran-3-carboxylic acid; |
| B-54 | 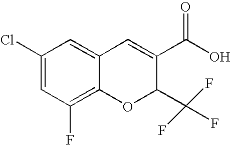 |
| | 6-chloro-8-fluoro-2-trifluoromethyl-2H-1-benzopyran-3-carboxylic acid; |
| B-55 | 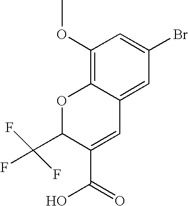 |
| | 6-bromo-8-methoxy-2-trifluoromethyl-2H-1-benzopyran-3-carboxylic acid; |
| B-56 | 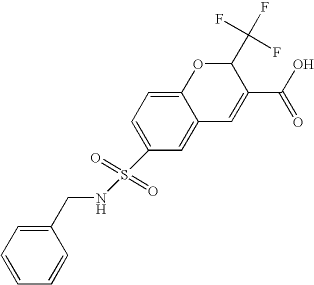 |
| | 6-[[(phenylmethyl)amino]sulfonyl]-2-trifluoromethyl-2H-1-benzopyran- |
| | 3-carboxylic acid; |
| B-57 | 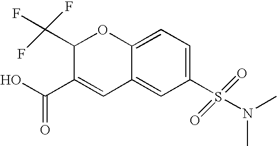 |
| | 6-[(dimethylamino)sulfonyl]-2-trifluoromethyl-2H-1-benzopyran- |
| | 3-carboxylic acid; |
| B-58 | 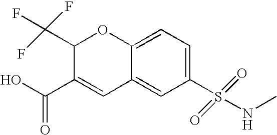 |
| | 6-methylamino)sulfonyl]-2-trifluoromethyl-2H-1-benzopyran- |
| | 3-carboxylic acid; |
| B-59 | 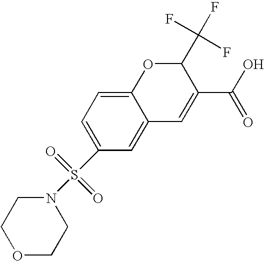 |
| | 6-[(4-morpholino)sulfonyl]-2-trifluoromethyl-2H-1-benzopyran- |
| | 3-carboxylic acid; |
| B-60 | 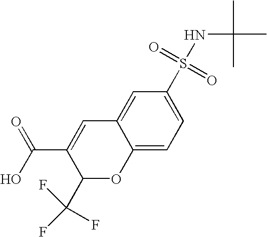 |
| | 1-dimethylethyl)aminosulfonyl]-2-trifluoromethyl- |
| | 2H-1-benzopyran-3-carboxylic acid; |
| B-61 | 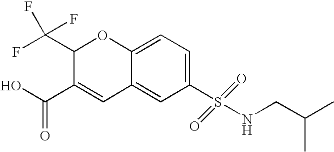 |
| | 6-[(2-methylpropyl)aminosulfonyl]-2-trifluoromethyl-2H-1-benzopyran- |
| | 3-carboxylic acid; |
| B-62 | 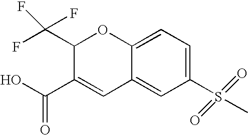 |
| | 6-methylsulfonyl-2-trifluoromethyl-2H-1-benzopyran-3-carboxylic acid; |
| B-63 | 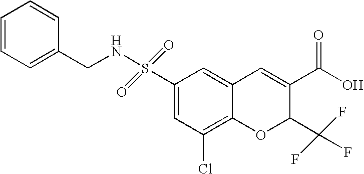 |
| | 8-chloro-6-[[(phenylmethyl)amino]sulfonyl]-2-trifluoromethyl- |
| | 2H-1-benzopyran-3-carboxylic acid; |
| B-64 | 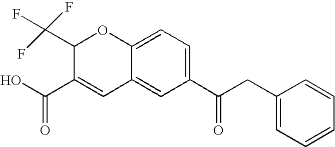 |
| | 6-phenylacetyl-2-trifluoromethyl-2H-1-benzopyran-3-carboxylic acid; |
| B-65 | 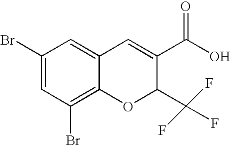 |
| | 6,8-dibromo-2-trifluoromethyl-2H-1-benzopyran-3-carboxylic acid; |
| B-66 | 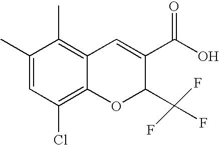 |
| | 8-chloro-5,6-dimethyl-2-trifluoromethyl-2H-1-benzopyran- |
| | 3-carboxylic acid; |
| B-67 | 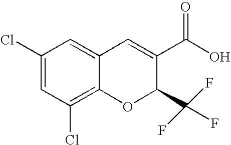 |
| | 6,8-dichloro-(S)-2-trifluoromcthyl-2H-1-benzopyran-3-carboxylic acid; |
| B-68 | 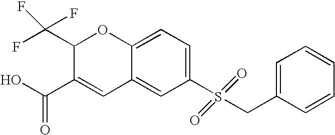 |
| | 6-benzylsulfonyl-2-trifluoromethyl-2H-1-benzopyran-3-carboxylic acid; |
| B-69 | 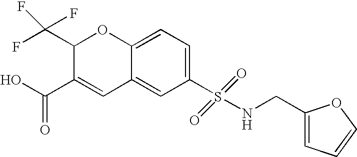 |
| | 6-[[-(2-furylmethyl)amino]sulfonyl]-2-trifluoromethyl- |
| | 2H-1-benzopyran-3-carboxylic acid; |
| B-70 | 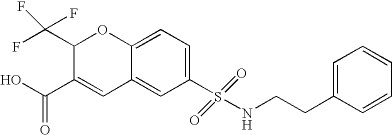 |
| | 6-[[N-(2-phenylethyl)amino]sulfonyl]-2-trifluoromethyl-2H-1- |
| | benzopyran-3-carboxylic acid; |
| B-71 | 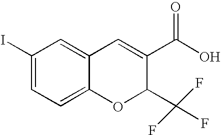 |
| | 6-iodo-2-trifluoromethyl-2H-1-benzopyran-3-carboxylic acid; |
| B-72 | 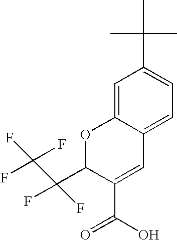 |
| | 7-(1,1-dimethylethyl)-2-pentafluoroethyl-2H- |
| | 1-benzopyran-3-carboxylic acid; |
| B-73 | 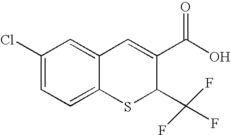 |
| | 6-chloro-2-trifluoromethyl-2H-1-benzothiopyran-3-carboxylic acid; |
| B-74 | 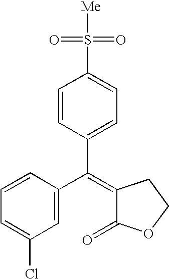 |
| | 3-[(3-chloro-phenyl)-(4-methanesulfonyl-phenyl)-methylene]- |
| | dihydro-furan-2-one or BMS-347070; |
| B-75 | 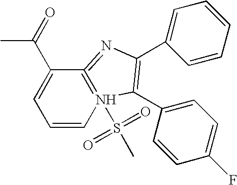 |
| | 8-acetyl-3-(4-fluorophenyl)-2-(4-methylsulfonyl)phenyl- |
| | imidazo(1,2-a)pyridine; |
| B-76 | 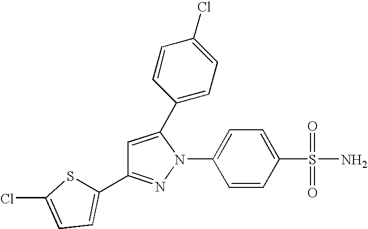 |
| | 5,5-dimethyl-4-(4-methylsulfonyl)phenyl-3-phenyl-2-(5H)-furanone; |
| B-77 | 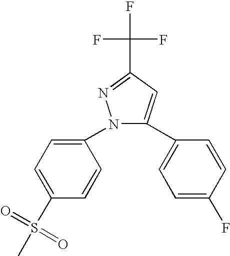 |
| | 5-(4-fluorophenyl)-1-[4-(methylsulfonyl)phenyl]- |
| | 3-(trifluoromethyl)pyrazole; |
| B-78 | 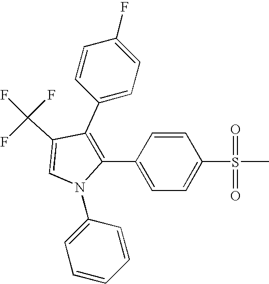 |
| | 4-(4-fluorophenyl)-5-[4-(methylsulfonyl)phenyl]- |
| | 1-phenyl-3-(trifluoromethyl)pyrazole; |
| B-79 | 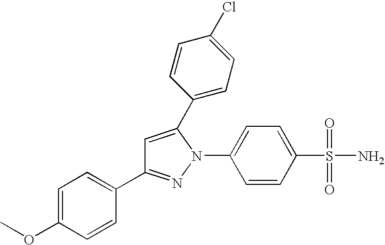 |
| | 4-(5-(4-chlorophenyl)-3-(4-methoxyphenyl)-1H-pyrazol-1-yl) |
| | benzenesulfonamide; |
| B-80 | 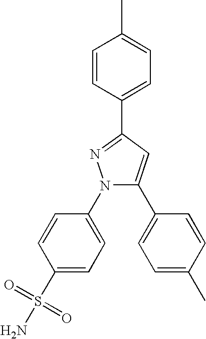 |
| | 4-(3,5-bis(4-methylphenyl)-1H-pyrazol-1-yl)benzenesulfonamide; |
| B-81 | 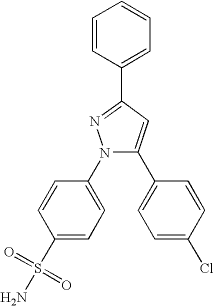 |
| | 4-(5-(4-chlorophenyl)-3-phenyl-1H-pyrazol-1-yl)benzenesulfonamide; |
| B-82 | 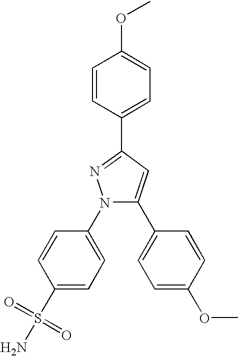 |
| | 4-(3,5-bis(4-methoxyphenyl)-1H-pyrazol-1-yl)benzenesulfonamide; |
| B-83 | 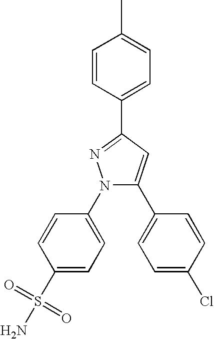 |
| | 4-(5-(4-chlorophenyl)-3-(4-methylphenyl)-1H-pyrazol- |
| | 1-yl)benzenesulfonamide; |
| B-84 | 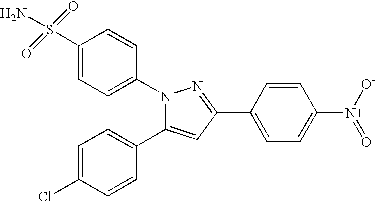 |
| | 4-(5-(4-chlorophenyl)-3-(4-nitrophenyl)-1H-pyrazol- |
| | 1-yl)benzenesulfonamide; |
| B-85 | 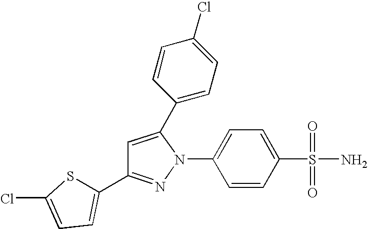 |
| | 4-(5-(4-chlorophenyl)-3-(5-chloro-2-thienyl)-1H-pyrazol- |
| | 1-yl)benzenesulfonamide; |
| B-86 | 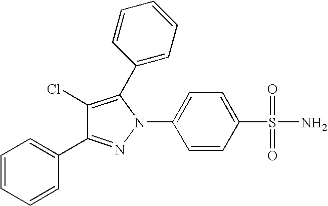 |
| | 4-(4-chloro-3,5-diphenyl-1H-pyrazol-1-yl)benzenesulfonamide; |
| B-87 | 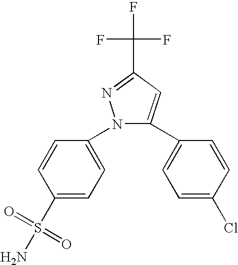 |
| | 4-[5-(4-chlorophenyl)-3-(trifluoromethyl)-1H-pyrazol- |
| | 1-yl]benzenesulfonamide; |
| B-88 | 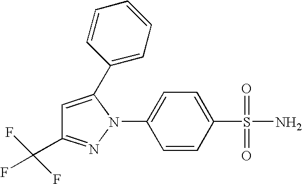 |
| | 4-[5-phenyl-3-(trifluoromethyl)-1H-pyrazol-1-yl]benzenesulfonamide; |
| B-89 | 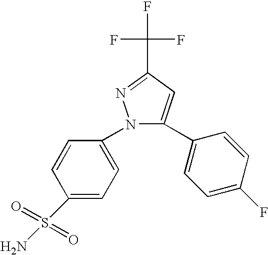 |
| | 4-[5-(4-fluorophenyl)-3-(trifluoromethyl)-1H-pyrazol- |
| | 1-yl]benzenesulfonamide; |
| B-90 | 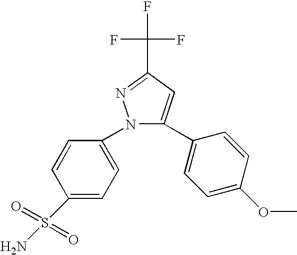 |
| | 4[5-(4-methoxyphenyl)-3-(trifluoromethyl)-1H-pyrazol- |
| | 1-yl]benzenesulfonamide; |
| B-91 | 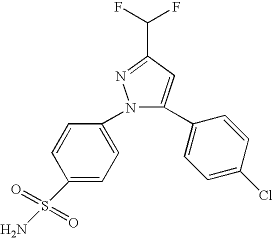 |
| | 4-[5-(4-chlorophenyl)-3-(difluoromethyl)-1H-pyrazol- |
| | 1-yl]benzenesulforiamide; |
| B-92 | 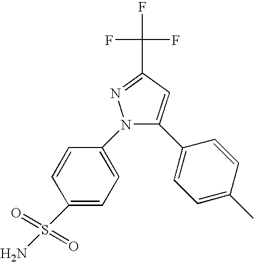 |
| | 4-[5-(4-methylphenyl)-3-(trifluoromethyl)-1H-pyrazol- |
| | 1-yl]benzenesulfonamide; |
| B-93 | 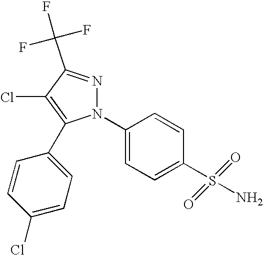 |
| | 4-[4-chloro-5-(4-chlorophenyl)-3-(trifluoromethyl)-1H-pyrazol-1-yl] |
| | benzenesulfonamide; |
| B-94 | 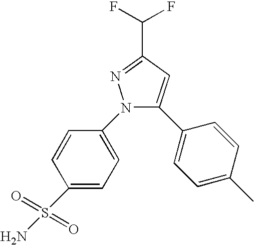 |
| | 4-[3-(difluoromethyl)-5-(4-methylphenyl)-1H-pyrazol- |
| | 1-yl]benzenesulfonamide; |
| B-95 | 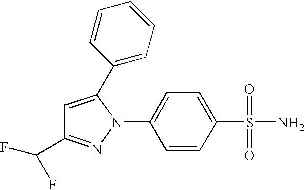 |
| | 4-[3-(difluoromethyl)-5-phenyl-1H-pyrazol-1-yl]benzenesulfonamide; |
| B-96 | 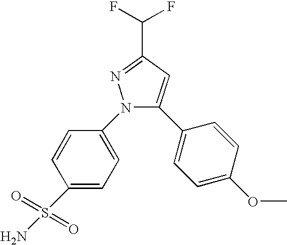 |
| | 4-[3-(difluoromethyl)-5-(4-methoxyphenyl)-1H-pyrazol- |
| | 1-yl]benzenesulfonamide; |
| B-97 | 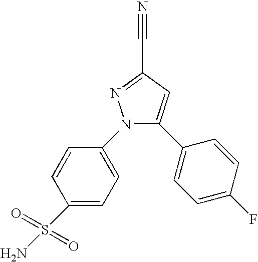 |
| | 4-[3-cyano-5-(4-fluorophenyyl-1H-pyrazol-1-yl]benzenesulfonamide; |
| B-98 | 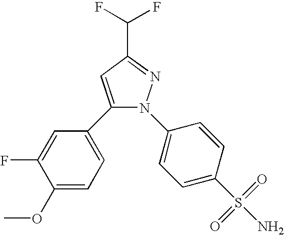 |
| | 4-[3-(difluoromethyl)-5-(3-fluoro-4-methoxyphenyl)-1H-pyrazol-1-yl] |
| | benzenesulfonamide; |
| B-99 | 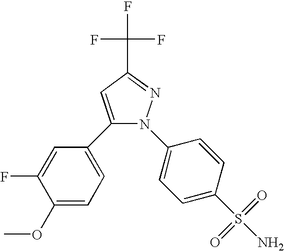 |
| | 4-[5-(3-fluoro-4-methoxyphenyl)-3-(trifluoromethyl)-1H-pyrazol-1-yl] |
| | benzenesulfonamide; |
| B-100 | 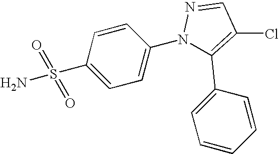 |
| | 4[4-chloro-5-phenyl-1H-pyrazol-1-yl]benzenesulfonamide; |
| B-101 | 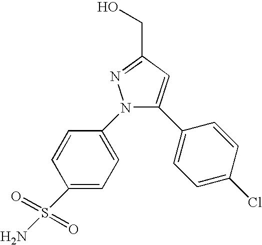 |
| | 4-[5-(4-chlorophenyl)-3-(hydroxymethyl)-1H-pyrazol- |
| | 1-yl]benzenesulfonamide; |
| B-102 | 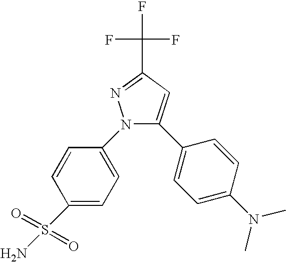 |
| | 4-[5-(4-(N,N-dimethylamino)phenyl)-3-(trifluoromethyl)- |
| | 1H-pyrazol-1-yl]benzenesulfonamide; |
| B-103 | 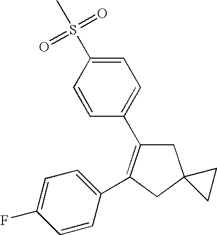 |
| | 5-(4-fluorophenyl)-6-∝-(methylsulfonyl)phenyl]spiro[2.4]hept-5-ene; |
| B-104 | 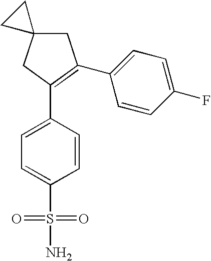 |
| | 4-[6-(4-fluorophenyl)spiro[2.4]hept-5-en-5-yl]benzenesulfonamide; |
| B-105 | 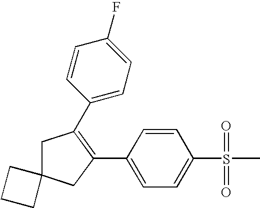 |
| | 6-(4-fluorophenyl)-7-[4-methylsulfonyl)phenyl[3.4]oct-6-ene; |
| B-106 | 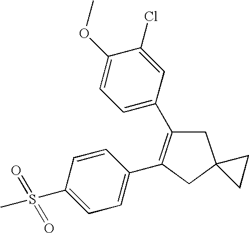 |
| | 5-(3-chloro-4-methoxyphenyl)-6-[4- |
| | (methylsulfonyl)phenyl]spiro[2.4]hept-5-ene; |
| B-107 | 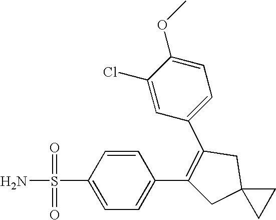 |
| | 4-[6-(3-chloro-4-methoxyphenyl)spiro[2.4]hept-5-en- |
| | 5-yl]benzensulfoamide, |
| B-108 | 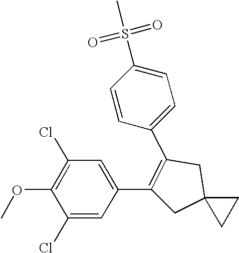 |
| | 5-(3,5-dichloro-4-methoxyphenyl)-6-[4-(methylsulfonyl)phenyl] |
| | spiro[2.4]hept-5-ene; |
| B-109 | 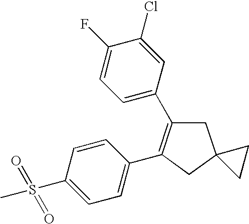 |
| | 5-(3-chloro-4-fluorophenyl)-6-[4- |
| | (methylsulfonyl)phenyl]spiro[2.4]hept-5-ene; |
| B-110 | 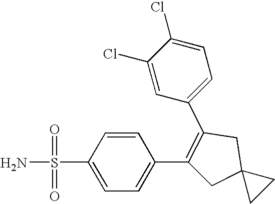 |
| | 4-[6-(3,4-dichlorophenyl)spiro[2.4]hept-5-en-5- |
| | yl]benzenesulfonamide |
| B-111 | 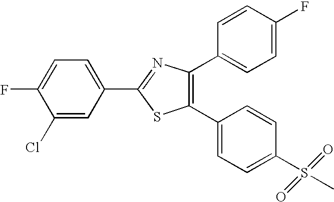 |
| | 2-(3-chloro-4-fluorophenyl)-4-(4-fluorophenyl)-5- |
| | (4-methylsulfonylphenyl)thiazole |
| B-112 | 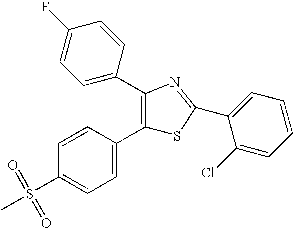 |
| | 2-(2-chlorophenyl)-4-(4-fluorophenyl)-5-(4-methylsulfonylphenyl)thiazole; |
| B-113 | 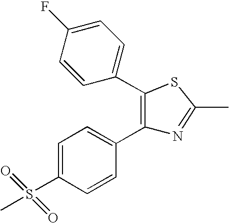 |
| | 5-(4-fluorophenyl)-4-(4-methylsulfonylphenyl)-2-methylthiazole |
| B-114 | 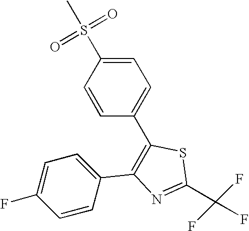 |
| | 4-(4-fluorophenyl)-5-(4-methylsulfonylphenyl)-2-methylthiazole |
| B-115 | 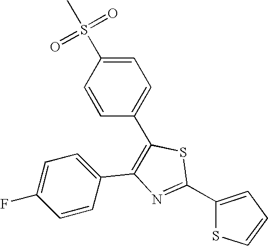 |
| | 4-(4-fluorophenyl)-4-(4-methylsulfonylphenyl)-2-(2-thienyl)thiazole |
| B-116 | 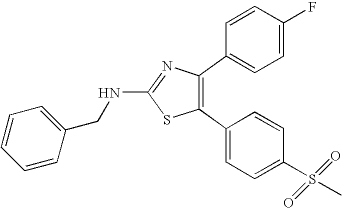 |
| | 4-(4-fluorophenyl)-5-(4-methylsulfonylphenyl)-2-benzylaminothiazole |
| B-117 | 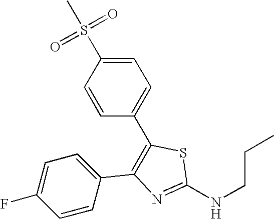 |
| | 4-(4-fluorophenyl)-5-(4-methylsulfonylphenyl)-2- |
| | (1-propylamino)thiazole; |
| B-118 | 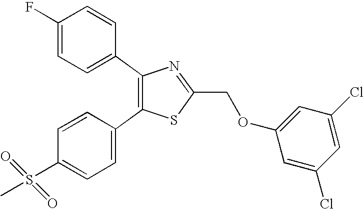 |
| | 2-((3,5-dichloropbenoxy)methyl)-4-(4-fluorophenyl)-5-[4- |
| | (methylsulfonyl)phenyl]thiazole; |
| B-119 | 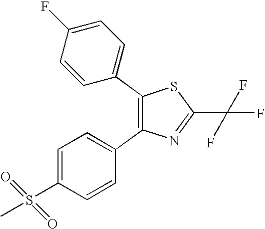 |
| | 5-(4-fluorophenyl)-4-(4-methylsulfonylphenyl)-2-trifluoromethylthiazole; |
| B-120 | 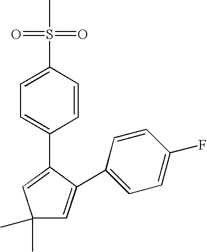 |
| | 1-methylsulfonyl-4-[1,1-dimethyl-4-(4-fluorophenyl) |
| | cyclopenta-2,4-dien-3-yl]benzene; |
| B-121 | 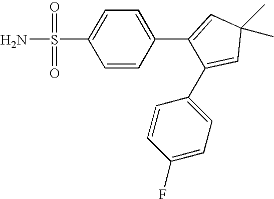 |
| | 4-[4-(4-fluorophenyl)-1,1-dimethylcyclopenta-2,4-dien-3-yl] |
| | benzenesulfonamide; |
| B-122 | 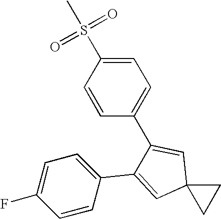 |
| | 5-(4-fluorophenyl)-6-[4-methylsulfonyl)phenyl]spiro[2.4]hepta-4,6-diene; |
| B-123 | 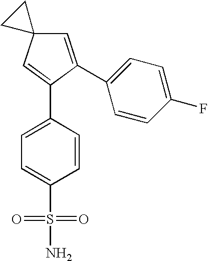 |
| | 4-[6-(4-fluorophenyl)spiro[2.4]hepta-4,6-dien-5-yl]benzenesulfonamide; |
| B-124 | 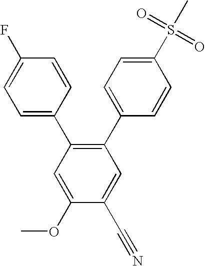 |
| | 6-(4-fluorophenyl)-2-methoxy-5-[4-(methylsulfonyl)phenyl]- |
| | pyridine-3-carbonitrile; |
| B-125 | 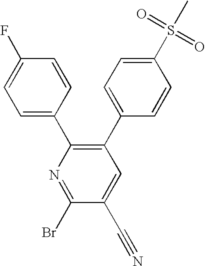 |
| | 2-bromo-6-(4-fluorophenyl)-5-[4-(methylsulfonyl)phenyl]- |
| | pyridine-3-carbonitrile; |
| B-126 | 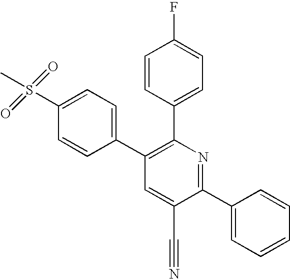 |
| | 6-(4-fluorophenyl)-5-[4-(methylsulfonyl)phenyl]-2-phenyl-pyndine- |
| | 3-carbonitrile; |
| B-127 | 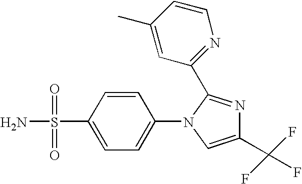 |
| | 4-[2-(4-methylpyridin-2-yl)-4-(trifluoromethyl)-1H-imidazo]-1-yl] |
| | benzenesulfonamide; |
| B-128 | 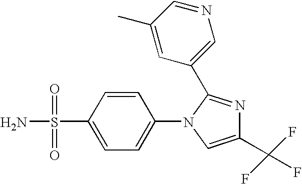 |
| | 4-[2-(5-methylpyridin-3-yl)-4-(trifluoromethyl)-1H-imidazol- |
| | 1-yl]benzenesulfonamide; |
| B-129 | 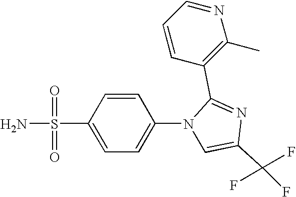 |
| | 4-[2-(2-methylpyridin-3-yl)-4-(trifluoromethyl)-1H-imidazol-1-yl] |
| | benzenesulfonamide; |
| B-130 | 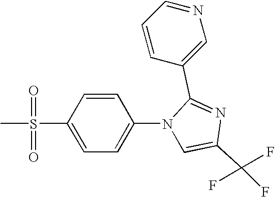 |
| | 3-[1-[4-(methylsulfonyl)phenyl]-4-(trifluoromethyl)- |
| | 1H-imidazol-2-yl]pyridine; |
| B-131 | 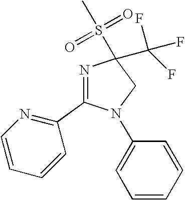 |
| | 2-[1-[4-(methylsulfonyl)phenyl-4-(trifluoromethyl)]- |
| | 1H-imidazol-2-yl]pyridine; |
| B-132 | 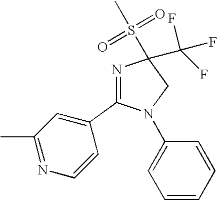 |
| | 2-methyl-4-[1-[4-(methylsulfonyl)phenyl-4-(trifluoromethyl)]- |
| | 1H-imidazol-2-yl]pyridine; |
| B-133 | 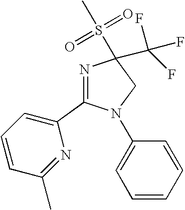 |
| | 2-methyl-6-[1-[4-(methylsulfonyl)phenyl-4-(trifluoromethyl)]- |
| | 1H-imidazol-2-yl]pyridine; |
| B-134 | 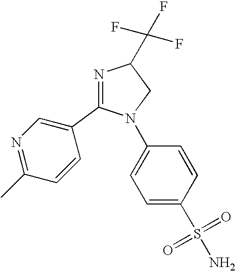 |
| | 4-[2-(6-methylpyridin-3-yl)-4-(trifluoromethyl)-1H-imidazol- |
| | 1-yl]benzenesulfonamide; |
| B-135 | 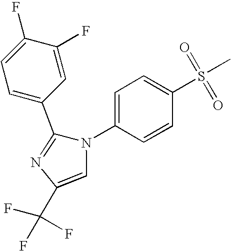 |
| | 2-(3,4-difluorophenyl)-1-[4-(methylsulfonyl)phenyl]- |
| | 4-(trifluoromethyl)-1H-imidazole; |
| B-136 | 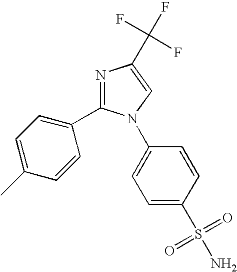 |
| | 4-[2-(4-methylphenyl)-4-(trifluoromethyl)-1H-imidazol- |
| | 1-yl]benzenesulfonamide; |
| B-137 | 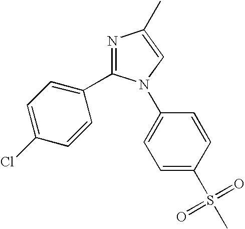 |
| | 2-(4-chlorophenyl)-4-[4-(methylsulfonyl)phenyl]-4-methyl-1H-imidazole; |
| B-138 | 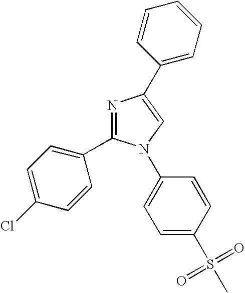 |
| | 2-(4-chlorophenyl)-1-[4-(methylsulfonyl)phenyl]-4-phenyl-1H-imidazole; |
| B-139 | 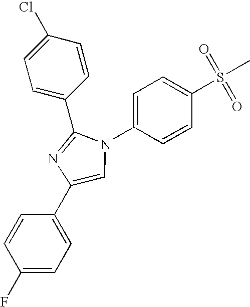 |
| | 2-(4-chlorophenyl)-4-(4-fluorophenyl)-1-∂4-(methylsulfonyl)phenyl]- |
| | 1H-imidazole; |
| B-140 | 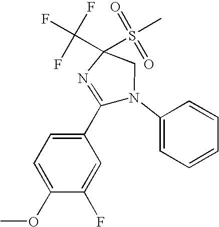 |
| | 2-(3-fluoro-4-methoxyphenyl)-1-[4-(methylsulfonyl)phenyl- |
| | 4-(trifluoromethyl)]-1H-imidazole; |
| B-141 | 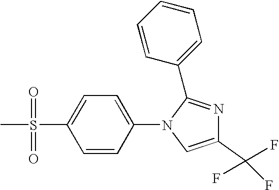 |
| | 1-[4-(methylsulfonyl)phenyl]-2-phenyl-4-trifluoromethyl-1H-imidazole; |
| B-142 | 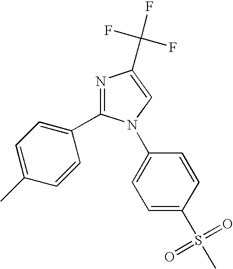 |
| | 2-(4-methylphenyl)-1-[4-(methylsulfonyl)phenyl]-4-trifluoromethyl- |
| | 1H-imidazole; |
| B-143 | 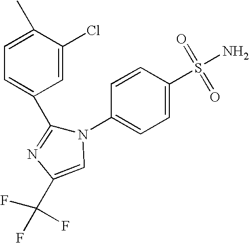 |
| | 4-[2-(3-chloro-4-methylphenyl)-4-(trifluoromethyl)- |
| | 1H-imidazol-1-yl]benzenesulfonamide; |
| B-144 | 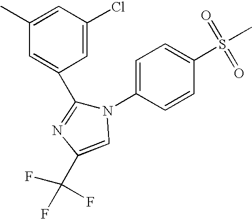 |
| | 2-(3-fluoro-5-methylphenyl)-1-[4-(methylsulfonyl)phenyl]- |
| | 4-(trifluoromethyl)-1H-imidazole; |
| B-145 | 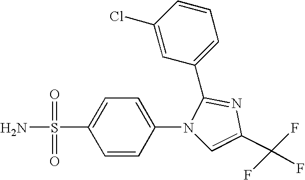 |
| | 4-[2-(3-fluoro-5-methylphenyl)-4-(trifluoromethyl- |
| | 1H-imidazole-1-yl]benzenesulfonamide. |
| B-146 | 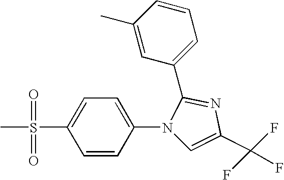 |
| | 2-(3-methylphenyl)-1-[4-(methylsulfonyl)phenyl]-4-trifluoromethyl- |
| | 1H-imidazole; |
| B-147 | 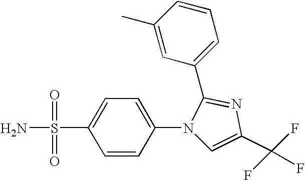 |
| | 4-[2-(3-methylphenyl)-4-trifluoromethyl-1H-imidazol- |
| | 1-yl]benzenesulfonamide; |
| 3-148 | 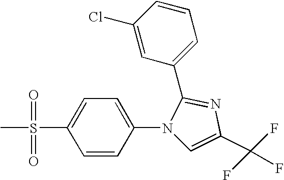 |
| | 1-[4-(methylsulfonyl)phenyl]-2-(3-chlorophenyl)-4-trifluoromethyl- |
| | 1H-imidazole |
| B-149 | 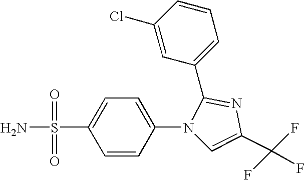 |
| | 4-[2-(3-chlorophenyl)-4-trifluoromethyl-1H-imidazol- |
| | 1-yl]benzenesulfonamide; |
| B-150 | 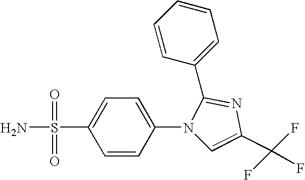 |
| | 4-[2-phenyl-4-trifluoromethyl-1H-imidazol-1-yl]benzenesulfonamide; |
| B-151 | 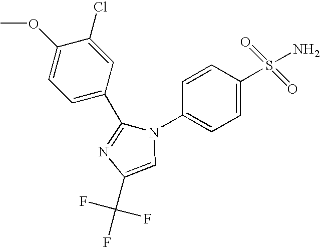 |
| | 4-[2-(4-methoxy-3-chlorophenyl)-4-trifluoromethyl-1H-imidazol- |
| | 1-yl]benzenesulfonamide; |
| B-152 | 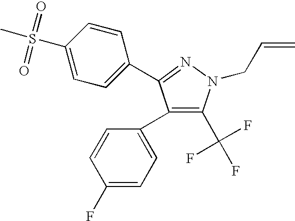 |
| | 1-allyl-4-(4-fluorophenyl)-3-[4-(methylsulfonyl)phenyl]- |
| | 5-(trifluoromethyl)-1H-pyrazole; |
| B-153 | 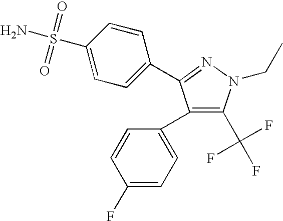 |
| | 4-[1-ethyl-4-(4-fluorophenyl)-5-(trifluoromethyl)-1H-pyrazol-3-yl] |
| | benzenesulfonamide; |
| B-154 | 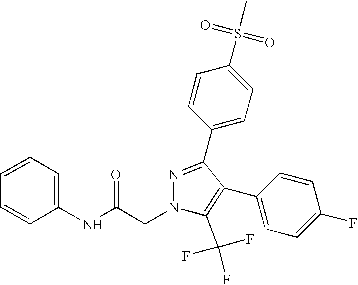 |
| | N-phenyl-[4-(4-fluorophenyl)-3-[4-(methylsulfonyl)phenyl]- |
| | 5-(trifluoromethyl)-1H-pyrazol-1-yl]acetamide; |
| B-155 | 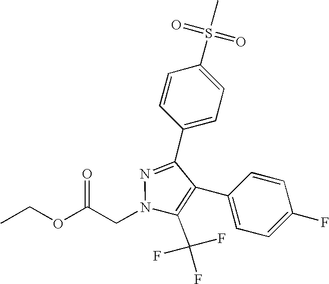 |
| | ethyl[4-(4-fluorophenyl)-3-[4-(methylsulfonyl)phenyl]- |
| | 5-(trifluoromethyl)-1H-pyrazol-1-yl]acetate; |
| B-156 | 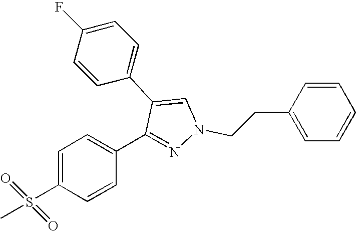 |
| | 4-(4-fluorophenyl)-3-[4-(methylsulfonyl)pbenyl]-1-(2-phenylethyl)- |
| | 1H-pyrazole; |
| B-157 | 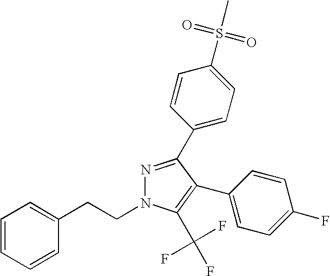 |
| | 4-(4-fluorophenyl)-3-[4-(methylsulfonyl)phenyl]- |
| | 1-(2-phenylethyl)-5-(trifluoromethyl)pyrazole; |
| B-158 | 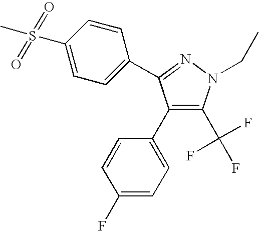 |
| | 1-ethyl-4-(4-fluorophenyl)-3-[4-methylsulfonyl)phenyl]- |
| | 5-(trifluoromethyl)-1H-pyrazole; |
| B-159 | 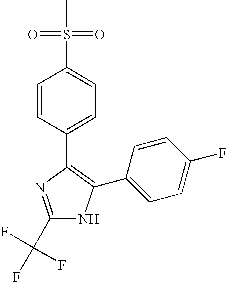 |
| | 5-(4-fluorophenyl)-4-(4-methylsulfonylphenyl)- |
| | 2-trifluoromethyl-1H-imidazole; |
| B-160 | 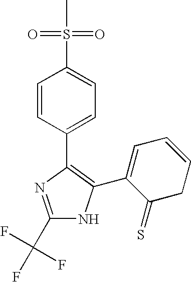 |
| | 4-[4-(methylsulfonyl)phenyl]-5-(2-thiophenyl)-2- |
| | (trifluoromethyl)-1H-imidazole; |
| B-161 | 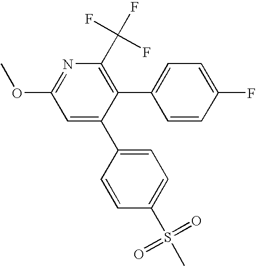 |
| | 5-(4-fluorophenyl)-2-methoxy-4-[4-(methylsulfonyl)phenyl]-6- |
| | (trifluoromethyl)pyridine; |
| B-162 | 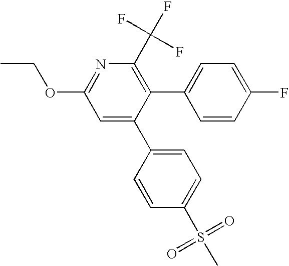 |
| | 2-ethoxy-5-(4-fluorophenyl)-4-[4-(methylsulfonyl)phenyl]- |
| | 6-(trifluoromethyl)pyridine; |
| B-163 | 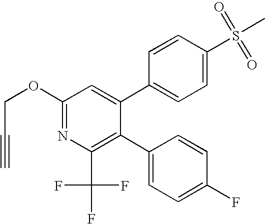 |
| | 5-(4-fluorophenyl)-4-[4-(methylsulfonyl)phenyl]- |
| | 2-(2-propynyloxy)-6-(trifluoromethyl)pyridine; |
| B-164 | 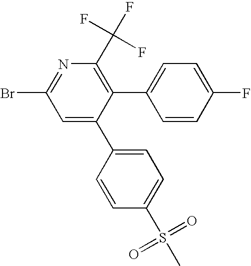 |
| | 2-bromo-5-(4-fluorophenyl)-4-[4-(methylsulfonyl)phenyl] |
| | 6-(trifluoromethyl)pyridine; |
| B-165 | 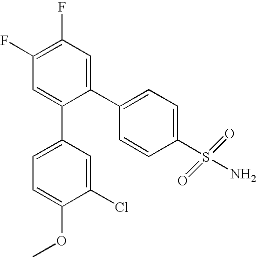 |
| | 4-[2-(3-chloro-4-methoxyphenyl)-4,5-difluorophenyl]benzenesulfonamide; |
| B-166 | 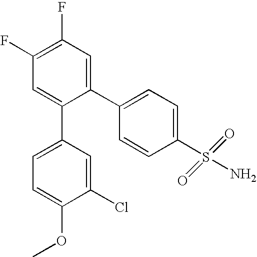 |
| | 1-(4-fluorophenyl)-2-[4-methylsulfonyl)phenyl]benzene; |
| B-167 | 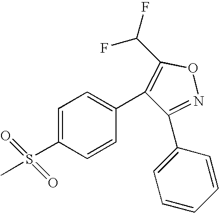 |
| | 5-difluoromethyl-4-(4-methylsulfonylphenyl)-3-phenylisoxazole; |
| B-168 | 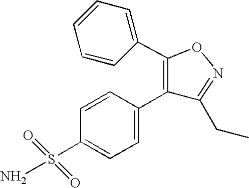 |
| | 4-[3-ethyl-5-phenylisoxazol-4-yl]benzenesulfonamide; |
| B-169 | 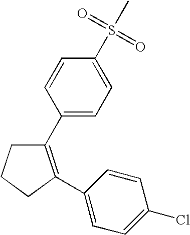 |
| | 4-[5-difluoromethyl-3-phenylisoxazol-4-yl]benzenesulfonamide; |
| B-170 | 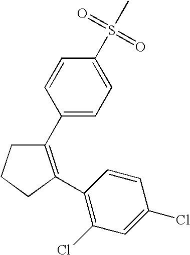 |
| | 4-[5-hydroxymethyl-3-phenylisoxazol-4-yl]benzenesulfonamide; |
| B-171 | 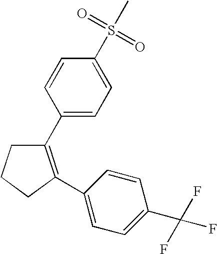 |
| | 4-[5-methyl-3-phenyl-isoxazol-4-yl]benzenesulfonamide; |
| B-172 | 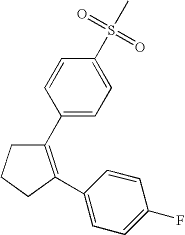 |
| | 1-[2-(4-fluorophenyl)cyclopenten-1-yl]-4-(methylsulfonyl)benzene; |
| B-173 | 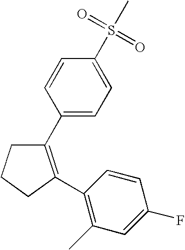 |
| | 1-[2-(4-fluoro-2-methylphenyl)cyclopenten-1-yl]-4- |
| | (metbylsulfonyl)benzene; |
| B-174 | 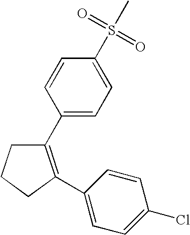 |
| | 1-[2-(4-chlorophenyl)cyclopenten-1-yl]-4-(methylsulfonyl)benzene; |
| B-175 | 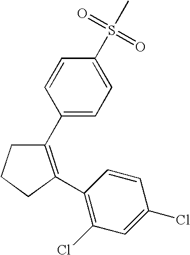 |
| | 1-[2-(2,4-dichlorophenyl)cyclopenten-1-yl]-4-(methylsulfonyl)benzene; |
| B-176 | 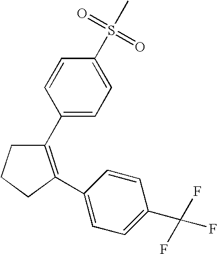 |
| | 1-[2-(4-trifloromethylphenyl)cyclopenten-1-yl]-4-(methylsulfonyl)benzene; |
| B-177 | 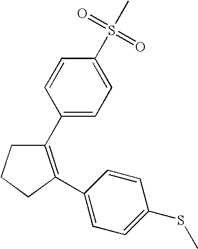 |
| | 1-[2-(4-methylthiophenyl)cyclopenten-1-yl]-4-(methylsulfonyl)benzene; |
| B-178 | 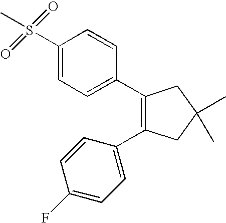 |
| | 1-[2-(4-fluorophenyl)-4,4-dimethylcyclopenten-1-yl]-4- |
| | (methylsulfonyl)benzene; |
| B-179 | 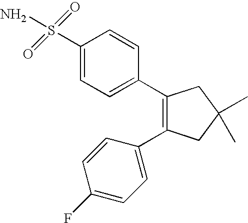 |
| | 4-[2-(4-fluorophenyl)-4,4-dimethylcyclopenten-1-yl]benzenesulfonamide; |
| B-180 | 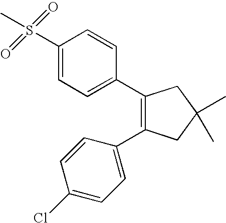 |
| | 1-[2-(3-chlorophenyl)-4,4-dimethylcyclopenten-1-yl]-4- |
| | (methylsulfonyl)benzene; |
| B-181 | 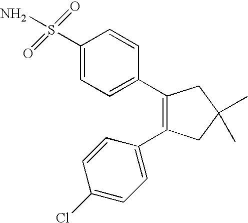 |
| | 4-[2-(4-chlorophenyl)-4,4-dimethylcyclopenten-1-yl]benzenesulfonamide; |
| B-182 | 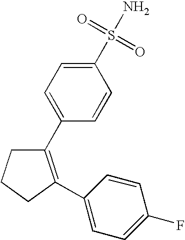 |
| | 4-[2-(4-fluorophenyl)cyclopenten-1-yl]benzenesulfonamide; |
| B-183 | 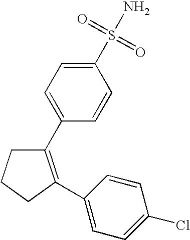 |
| | 4-[2-(4-chlorophenyl)cyclopenten-1-yl]benzenesulfonamide; |
| B-184 | 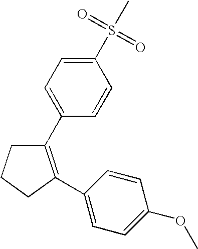 |
| | 1-[2-(4-methoxyphenyl)cyclopenten-1-yl)-4-(methylsulfonyl)benzene; |
| B-185 | 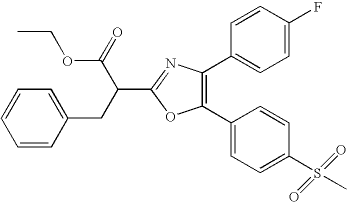 |
| | 1-[2-(2,3-difluorophenyl)cyclopenten-1-yl]-4-(methylsulfonyl)benzene; |
| B-186 | 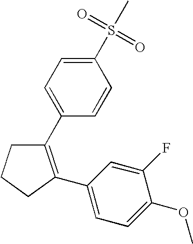 |
| | 4-[2-(3-fluoro-4-methoxyphenyl)cyclopenten-1-yl]benzenesulfonamide; |
| B-187 | get,0183 |
| | 1-[2-(3-chloro-4-methoxyphenyl)cyclopenten-1-yl]-4- |
| | (methylsulfonyl)benzene; |
| B-188 | 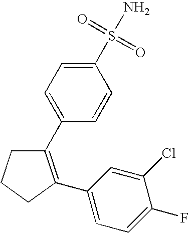 |
| | 4-[2-(3-chloro-4-fluorophenyl)cyclopenten-1-yl]benzenesulfonamide; |
| B-189 | 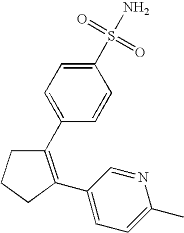 |
| | 4-[2-(2-methylpyridin-5-yl)cyclopenten-1-yl]benzenesulfonamide; |
| B-190 | 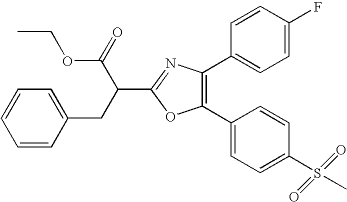 |
| | ethyl 2-[4-(4-fluorophenyl)-5-[4-(methylsulfonyl)phenyl]oxazol-2-yl]- |
| | 2-benzyl-acetate, |
| B-191 | 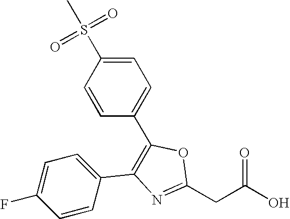 |
| | 2-[4-(4-fluorophenyl)-5-[4-(methylsulfonyl)phenyl]oxazol-2-yl]acetic acid; |
| B-192 | 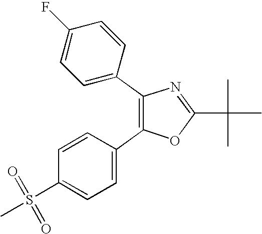 |
| | 2-(tert-butyl)-4-(4-fluorophenyl)-5-[4-(methylsulfonyl)phenyl]oxazole; |
| B-193 | 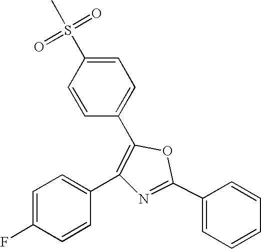 |
| | 4-(4-fluorophenyl)-5-[4-(methylsulfonyl)phenyl]-2-phenyloxazole; |
| B-194 | 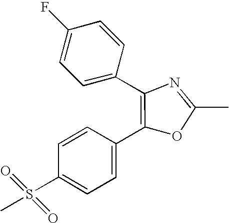 |
| | 4-(4-fluorophenyl)-2-methyl-5-[4-(methylsulfonyl)phenyl]oxazole; |
| B-195 | 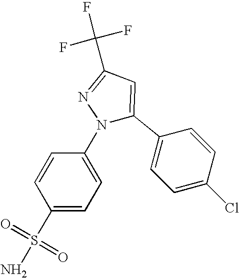 |
| | 4-[5-(3-fluoro-4-methoxyphenyl)-2-trifluoromethyl- |
| | 4-oxazolyl]benzenesulfonamide; |
| B-196 | 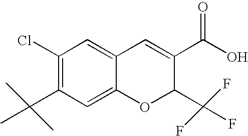 |
| | 6-chloro-7-(1,1-dimethylethyl)-2-trifluoromethyl-2H- |
| | 1-benzopyran-3-carboxylic acid; |
| B-197 | 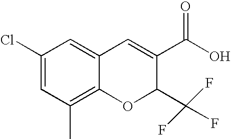 |
| | 6-chloro-8-methyl-2-trifluoromethyl-2H-1-benzopyran-3-carboxylic acid; |
| B-198 | 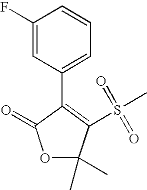 |
| | 5,5-dimethyl-3-(3-fluorophenyl)-4-methylsulfonyl-2(5H)-furanone; |
| B-199 | 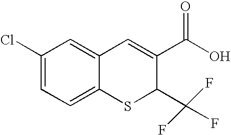 |
| | 6-chloro-2-trifluoromethyl-2H-1-benzothiopyran-3-carboxylic acid; |
| B-200 | 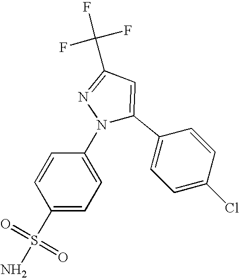 |
| | 4-[5-(4-chlorophenyl)-3-(trifluoromethyl)-1H-pyrazol- |
| | 1-yl]benzenesulfonamide; |
| B-201 | 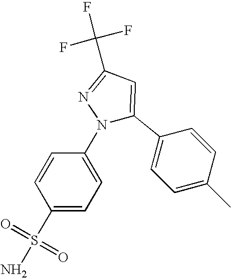 |
| | 4-[5-(4-methylphenyl)-3-(trifluoromethyl)-1H-pyrazol- |
| | 1-yl]benzenesulfonamide; |
| B-202 | 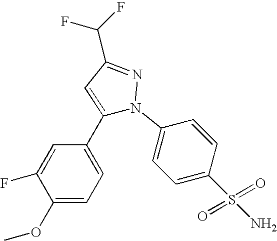 |
| | 4-[5-(3-fluoro-4-methoxyphenyl)-3-(difluoromethyl)- |
| | 1H-pyrazol-1-yl]benzenesulfonamide; |
| B-203 | 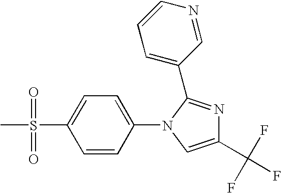 |
| | 3-[1-[4-(methylsulfonyl)phenyl]-4-trifluoromethyl- |
| | 1H-imidazol-2-yl]pyridine; |
| D-204 | 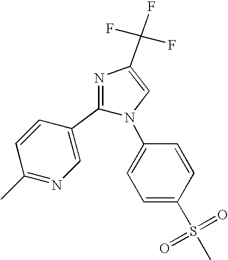 |
| | 2-methyl-5-[1-[4-(methylsulfonyl)phenyl]-4-trifluoromethyl- |
| | 1H-imidazol-2-yl]pyridine; |
| B-205 | 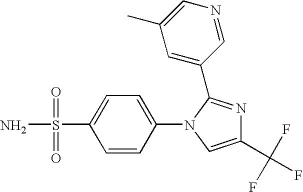 |
| | 4-[2-(5-methylpyridin-3-yl)-4-(trifluoromethyl)- |
| | 1H-imidazol-1-yl]benzenesulfonamide; |
| B-206 | 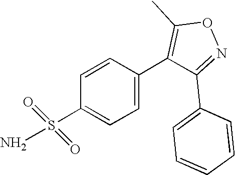 |
| | 4-[5-methyl-3-phenylisoxazol-4-yl]benzenesulfonamide; |
| B-207 | 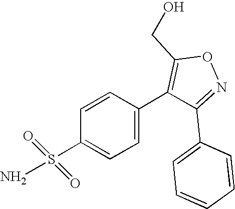 |
| | 4-[5-hydroxymethyl-3-phenylisoxazol-4-]benzenesulfonamide; |
| B-208 | 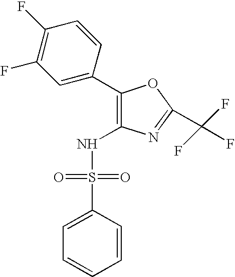 |
| | [2-trifluorometbyl-5-(3,4-difluorophenyl)-4-oxazolyl]benzenesulfonamide; |
| B-209 | 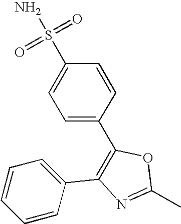 |
| | 4-[2-methyl-4-phenyl-5-oxazolyl]benzenesulfonamide; |
| B-210 | 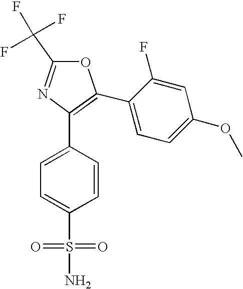 |
| | 4-[5-(2-fluoro-4-methoxyphenyl)-2-trifluoromethyl- |
| | 4-oxazolyl]benzenesulfonamide; |
| B-211 | 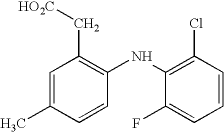 |
| B-212 | 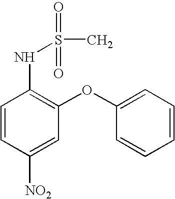 |
| | N-(4-nitro-2-phenoxy-phenyl)-methanesulfonamide or Nimesulide |
| B-213 | 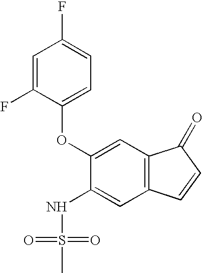 |
| | N-[6-(2,4-difluoro-phenoxy)-1-oxo-inden-5-yl]-methanesulfonamide |
| | or Flosulide |
| B-214 | 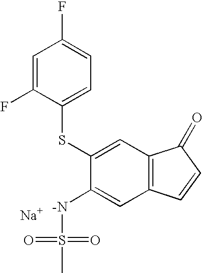 |
| | N-[6-(2,4-difluoro-phenylsulfanyl)-1-oxo-1H-inden-5-yl]- |
| | methanesulfonamide, soldium salt, or L-745337 |
| B-125 | 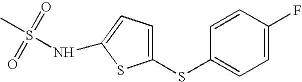 |
| | N-[5-(4-fluoro-phenylsulfanyl]-thiophen-2-yl]- |
| | methanesulfonamide or RWJ-63556 |
| B-216 | 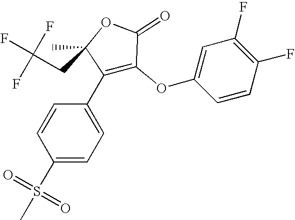 |
| | 3-(3,4-difluoro-phenoxy)-4-(4-methanesulfonyl-phenyl)-5-methyl- |
| | 5-(2,2,2-trifluoro-ethyl)-5H-furan-2-one or L-784512 |
| B-217 | 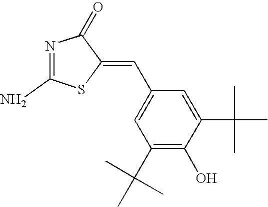 |
| | (5Z)-2-amino-5-[[3,5-bis(1,1-dimethylethyl)-4-hydroxyphenyl]methylene]- |
| | 4(5H)-thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 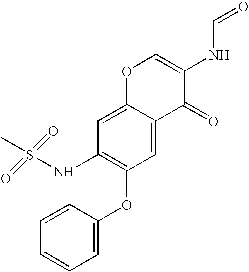 |
| | N-[3-(formylamino)-4-oxo-6-phenoxy-4H-1-benzopyran-7-yl]- |
| | methanesulfonamide or T614 |
| B-225 | D-1367 |
| P-226 | L-748731 |
| B-227 | 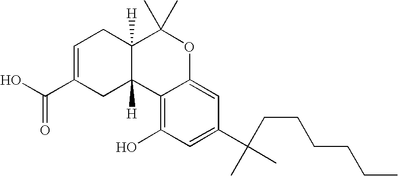 |
| | (6aR,10aR)-3-(1,1-dimethylheptyl)-6a,7,10,10a-tetrahydro-1-hydroxy- |
| | 6,6-dimethyl-6H-dibenzo[b,d]pyran-9-carboxylic acid or CT3 |
| B-228 | CGP-28238 |
| B-229 | 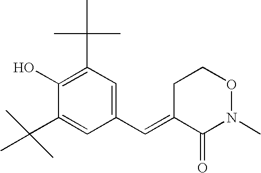 |
| | 4-[[3,5-bis(1,1-dimethylethyl)-4-hydroxyphenyl]methylene] |
| | dihydro-2-methyl-2H-1,2-oxazin-3(4H)-one or BF-389 |
| B-230 | GR-253035 |
| B-231 | 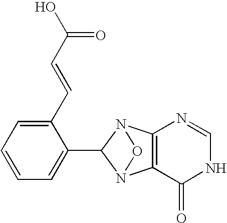 |
| | 2-(6-dioxo-9H-purin-8-yl)cinnamic acid |
| B-232 | S-2474 |
| B233 | 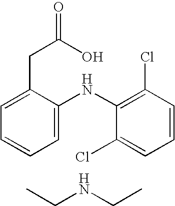 |

In addition to the COX-2 selective inhibitor, the composition also comprises N-acetyl-p-aminophenol, commonly known as acetaminophen, which has the following structure:

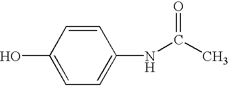

Acetaminophen is also known by the following trade or chemical names, all of which are suitable for use in the present invention: 4-acetamidophenol; Tylenol®; paracetamol; acetaminofen; panadol®; valgesic; acetagesic; N-(4-hydroxyphenyl)acetamide; p-Acetylaminophenol; 4′-hydroxyacetanilide; p-hydroxyacetanilide; tempra®; anacin-3; darvocet-n; datril; dristan®; liquiprin; percogesic®; phenaphen®; sinutab®; acamol; anhiba; ben-u-ron; bickie-mol; dial-a-gesic; doliprane; temlo; valadol; cetadol; acetalgin; enelfa; exdol; finimal; dirox; nobedon; tabalgin; calpol; dymadon; momentum®; naprinol; panets; homoolan; febrilix; abensanil; anaflon; amadil; eneril; tralgon; hedex; pacemo; panex; panofen; parmol; lyteca; apamide; tapar; gelocatil; korum; paraspen; dafalgan; disprol; dolprone; anuphen; apap; arthralgen; bancap®; capital®; proxyphene/acetamine; dularin; elixodyne; liqiprine; sk-apap; algotropyl; alpinyl; alvedon; anelix; apadon; clixodyne; febro-gesic; febrolin; fendon; G 1; janupap; lestemp; liquagesic; lonarid; lyteca syrup; multin; NAPA; napafen; napap; parapan; pedric; phendon; pyrinazine; tempanal; tussapap; calpol infant; paldesic; analeve; salzone; alpiny; p-acetaminophenol.

It is also contemplated that derivatives of acetaminophen may be used in the practice of the invention. Generally speaking, particularly suitable derivatives of acetaminophen may have an increased half-life, a slower rate of clearance from the body, increased bioactivity, reduced toxicity or any combination of these properties. By way of example, a class of glycoside derivatives of acetaminophen described in U.S. Pat. No. 5,693,767, (which is hereby incorporated by reference in its entirety) exhibit increased water solubility properties. A further embodiment employs carbonyl derivatives of acetaminophen as more fully described in U.S. Pat. No. 5,103,021, which is hereby incorporated by references in its entirety. Other suitable derivatives include prodrugs and salts of acetaminophen, such as the alkali metal and alkaline-earth salts of acetaminophen described in U.S. Pat. No. 6,160,020, which is hereby incorporated by reference in its entirety.

The COX-2 selective inhibitors and acetaminophen useful in the practice of the present methods can be formulated into pharmaceutical compositions and administered by any means that will deliver a therapeutically effective dose. These compositions, for example, can be, if appropriate, administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form will vary depending upon the subject to be treated, the particular inflammation, inflammation-mediated condition, or pain condition being treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated and the particular purpose to be achieved according to the determination of the skilled artisan.

Generally speaking, however, the pharmaceutical compositions typically contain a COX-2 selective inhibitor in the range of about 0.1 to 2000 milligrams, preferably in the range of about 0.5 to 500 milligrams and most preferably between about 1 and 200 milligrams. A daily dose of about 0.01 to 100 milligrams per killigram body weight, more preferably between about 0.1 and about 50 milligrams per killigram body weight and most preferably from about 1 to 20 milligrams per killigram body weight, may be appropriate. Moreover, the pharmaceutical compositions may contain acetaminophen in the range of about 0.1 to 7000 milligrams, preferably in the range of about 0.5 to 4000 milligrams, and most preferably in the range of about 1000 to 2000 milligrams.

The timing of the administration of the cyclooxygenase-2 selective inhibitor in relation to the administration of acetaminophen may vary from subject to subject and depend upon the particular condition being treated. In one embodiment of the invention, the cyclooxygenase-2 selective inhibitor and acetaminophen may be administered substantially simultaneously, meaning that both agents may be administered to the subject at approximately the same time. For example, the cyclooxygenase-2 selective inhibitor or pharmaceutically acceptable salt or prodrug thereof is administered during a continuous period beginning on the same day as the beginning of acetaminophen administration and extending to a period after the end of acetaminophen administration. Alternatively, the cyclooxygenase-2 selective inhibitor and acetaminophen may be administered sequentially, meaning that they are administered at separate times during separate treatments. In one such embodiment, for example, the cyclooxygenase-2 selective inhibitor or a pharmaceutically acceptable salt or prodrug thereof is administered during a continuous period beginning prior to administration of acetaminophen and ending after administration of the acetaminophen. Of course, it is also possible that the cyclooxygenase-2 selective inhibitor may be administered either more or less frequently than the acetaminophen. One skilled in the art can readily design suitable treatment regiments for a particular subject depending on the particular inflammation condition being treated. Moreover, it will be apparent to those skilled in the art that it is possible, and perhaps desirable, to combine various times and methods of administration in the practice of the present invention.

The composition of the invention may be employed to prevent or treat a subject having, or at risk for developing, a condition which is mediated in whole or in part by prostaglandins and in particular, mediated by COX-2. By way of example, these conditions include, but are not limited to, the treatment of inflammation in a subject, and for treatment of other inflammation-mediated disorders, such as, an analgesic in the treatment of pain and headaches, or as an antipyretic for the treatment of fever. For example, the compositions of the invention would be useful to treat arthritis, including but not limited to rheumatoid arthritis, spondyloarthopathies, gouty arthritis, osteoarthritis, systemic lupus erythematosus and juvenile arthritis. Such compositions would be useful in the treatment of asthma, bronchitis, menstrual cramps, tendinitis, bursitis, skin-related conditions such as psoriasis, eczema, burns and dermatitis, and from post-operative inflammation including ophthalmic surgery such as cataract surgery and refractive surgery. Compositions of the invention also would be useful to treat gastrointestinal conditions such as inflammatory bowel disease, Crohn's disease, gastritis, irritable bowel syndrome and ulcerative colitis. The compositions would also be useful in the treatment of pulmonary inflammation, such as that associated with viral infections and cystic fibrosis. The compositions of the invention are useful as anti-inflammatory agents, such as for the treatment of arthritis, with the additional benefit of having significantly less harmful side effects. These compositions would also be beneficial in the treatment of allergic rhinitis, respiratory distress syndrome, endotoxin shock syndrome, atherosclerosis and central nervous system damage resulting from stroke, ischemia and trauma.

In yet another embodiment, the compositions may be employed for the treatment and prevention of cancer, including but not limited to the following types of cancer: colon, breast, prostate, bladder, or lung. The compositions may also be utilized as chemopreventive agents.

Additionally, the compositions would be beneficial for the treatment of certain central nervous system disorders such as cortical dementias including Alzheimer's disease.

Compositions of the invention would also be useful in treating inflammation in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

In yet another embodiment, the compositions are employed for the treatment and prevention of ophthalmic diseases, such as retinitis, retinopathies, uveitis, ocular photophobia, and of acute injury to the eye tissue.

Moreover, the compositions can be employed for the treatment and prevention of pain symptoms, including but not limited to postoperative pain, dental pain, muscular pain, pain resulting from cancer, myalgia, articular pain or neuralgia. These pain symptoms can either be the result of an inflammatory mediated response or can result from a non-inflammatory mediated response.

In addition to a cyclooxygenase-2 selective inhibitor and acetaminophen, the composition of the invention may also include any agent that attenuates pain or inflammation for the particular condition being treated. By way of example, such agents include aspirin and other NSAIDs and, in particular, when the condition is pain, opioid analgesics such as morphine. Specific anti-inflammatory agents include diclofenac, ibuprofen, indomethacin, ketoprofen, naproxen, piroxicam and sulindac. Suitable opioid analgesics of use in conjunction with a composition of the current invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Preferred salts of these opioid analgesics include morphine sulphate, morphine hydrochloride, morphine tartrate, codeine phosphate, codeine sulphate, dihydrocodeine bitartrate, dacetylmorphine hydrochloride, hydrocodone bitartrate, hydromorphone hydrochloride, levorphanol tartrate, oxymorphone hydrochloride, alfentanil hydrochloride, buprenorphine hydrochloride, butorphanol tartrate, fentanyl citrate, meperidine hydrochloride, methadone hydrochloride, nalbuphine hydrochloride, propoxyphene hydrochloride, propoxyphene napsylate (2-naphthalenesulphonic acid (1:1) monohydrate), and pentazocine hydrochloride.

### EXAMPLES

The following examples are intended to provide illustrations of the application of the present invention. The following examples are not intended to completely define or otherwise limit the scope of the invention.

### Example 1

### Rat Carrageenan Foot Pad Edema Test

The anti-inflammatory properties of COX-2 selective inhibitors for use, along with their combination with acetaminophen, in the present methods can be determined by the rat carrageenan footpad edema test. The carrageenan foot edema test is performed with materials, reagents and procedures essentially as described by Winter, et al., (Proc. Soc. Exp. Biol. Med., 111: 544, 1962). Male Sprague-Dawley rats are selected in each group so that the average body weight is as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later, a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDs, in Non-steroidal Anti-Inflammatory Drugs, (J. Lombardino, ed. 1985)). The percentage inhibition shows the percentage decrease from control paw volume determined in this procedure.

### Example 2

### Rat Plantar Test

The ability of COX-2 selective inhibitors along with acetaminophen for use in the method of the present invention to prevent hyperalgesia can be determined by the rat plantar test. The rat plantar test is performed with materials, reagents and procedures essentially as described by Hargreaves et al. (Pain. (1988) 32:77-88). Male Sprague-Dawley rats are selected in each group so that the average body weight is as close as possible. An inflammation is induced in the rats by intraplantar injection of an approximately 0.05% suspension of Mycobacterium butyricum. Six hours after this injection, a heat stimulus is applied by infrared ray onto the plantar face of the hind paw of the rat. The nociceptive reaction of the rat manifests itself by the withdrawal or the licking of the paw. The time of this pain reaction is then measured. Additionally the COX-2 selective inhibitor and acetaminophen are administered via the oral route approximately one hour before the plantar test. The average time of pain reaction in a group of drug-treated animals is then compared with that of a group of placebo-treated animals in order to determine the hyperalgesia preventative effect of the composition of the present invention.

### Example 3

### Phenylbenzoquinone Test

The analgesic properties of COX-2 selective inhibitors along with acetaminophen for use in the present methods can be determined by the phenylbenzoquinone test. The phenylbenzoquinone test is performed with the materials, reagents, and procedures essentially as described in Siegmund et al. (Proc. Sec. Exp. Biol. Med. (1957) 95:729-731). Male Sprague-Dawley rats are selected in each group so that the average body weight is as close as possible. One hour after the oral administration of the composition of the present invention, a 0.02% solution of phenylbenzoquinone is administered via the intraperitoneal route to each rat. The number of pain reactions, measured as abdominal torsions and stretches, is then counted between the fifth and sixth minute after injection of the phenylbenzoquinone. The average number of pain reactions in a group of drug-treated animals is then compared with that of a group of placebo-treated animals in order to determine the analgesic properties of the composition of the present invention.

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A composition comprising a phenyl acetic acid derivative cyclooxygenase-2 selective inhibitor or a pharmaceutically acceptable salt or prodrug thereof and acetaminophen or a pharmaceutically acceptable salt or prodrug thereof.

2. The composition of claim 1 wherein the cyclooxygenase-2 selective inhibitor or a pharmaceutically acceptable salt or prodrug thereof comprises a compound of the formula: 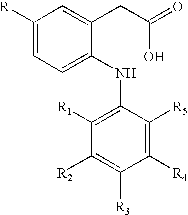
wherein:
R is methyl or ethyl;
R₁ is chloro or fluoro;
R₂ is hydrogen or fluoro
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl, provided that R₁, R₂, R₄ and R₅ are not all fluoro when R is ethyl and R₃ is H.

3. The composition of claim 2, wherein:
R is ethyl;
R¹ and R² are chloro;
R³ and R⁴ are hydrogen; and
and R⁵ is methyl.

4. The composition of claim 2 wherein the cyclooxygensase-2 selective inhibitor is selected from the group consisting of: 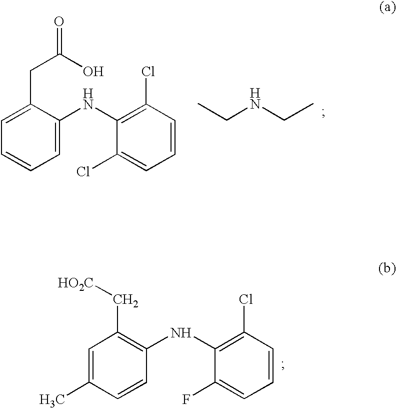
and any combination thereof.

5. A composition comprising a cyclooxygenase-2 selective inhibitor or a pharmaceutically acceptable salt or prodrug thereof and acetaminophen or a pharmaceutically acceptable salt or prodrug thereof, wherein the cyclooxygenase-2 selective inhibitor is selected from the group consisting of: 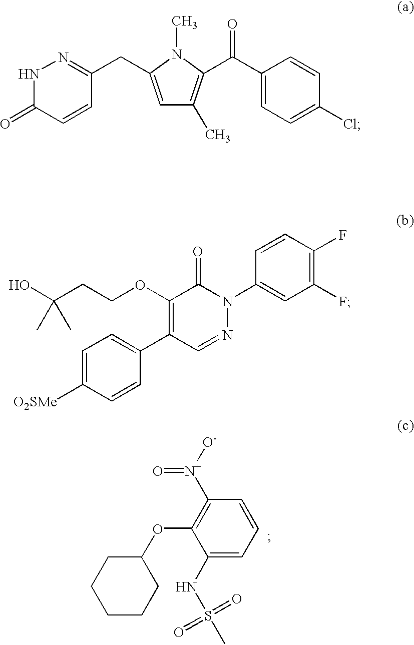
(d) a compound having the formula: 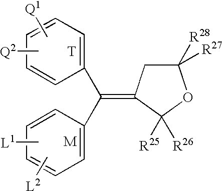
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or, Q¹ and Q² are methylenedioxy; or L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl, or oxygen.

6. A method for the treatment or prevention of inflammation, an inflammation-mediated disorder or pain in a subject, the method comprising administering to the subject a phenyl acetic acid derivative cyclooxygenase-2 selective inhibitor or a pharmaceutically acceptable salt or prodrug thereof and acetaminophen or a pharmaceutically acceptable salt or prodrug thereof.

7. The method of claim 6 wherein the cyclooxygenase-2 selective inhibitor or a pharmaceutically acceptable salt or prodrug thereof comprises a compound of the formula: 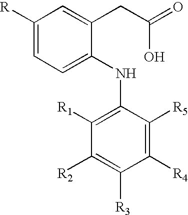
wherein:
R is methyl or ethyl;
R₁ is chloro or fluoro;
R₂ is hydrogen or fluoro
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl, provided that R₁, R₂, R₄ and R₅ are not all fluoro when R is ethyl and R₃ is H.

8. The method of claim 7 wherein:
R is ethyl;
R¹ and R² are chloro;
R³ and R⁴ are hydrogen; and
and R⁵ is methyl.

9. The method of claim 7 wherein the cyclooxygensase-2 selective inhibitor is selected from the group consisting of: 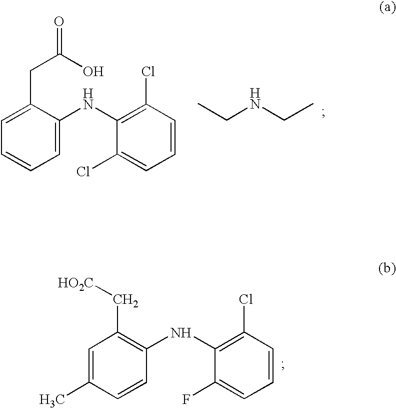
and any combination thereof.

10. A method comprising a cyclooxygenase-2 selective inhibitor or a pharmaceutically acceptable salt or prodrug thereof and acetaminophen or a pharmaceutically acceptable salt or prodrug thereof, wherein the cyclooxygenase-2 selective inhibitor is selected from the group consisting of: 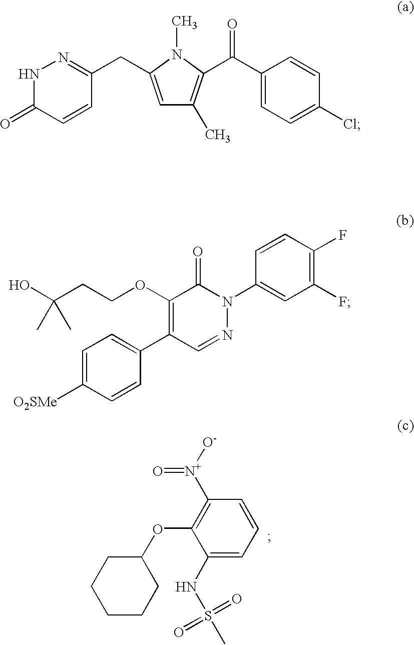
(d) a compound having the formula: 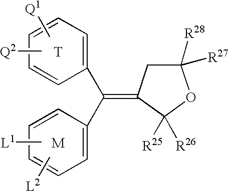
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or, Q¹ and Q² are methylenedioxy; or L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl, or oxygen.

11. The method of claim 6 wherein the prevention or treatment of inflammation, the inflammation mediated disorder or pain is achieved in part by substantial inhibition of prostaglandin synthesis.

12. The method of claim 6 wherein the inflammation mediated disorder is arthritis.

13. The method of claim 12 wherein the arthritis is rheumatoid arthritis.

14. The method of claim 12 wherein the arthritis is gouty arthritis.

15. The method of claim 12 wherein the arthritis is osteoarthritis.

16. The method of claim 12 wherein the arthritis is spondyloarthopathies.

17. The method of claim 12 wherein the arthritis is a symptom systemic lupus erythematosus.

18. The method of claim 12 wherein the arthritis is juvenile arthritis.

19. The method of claim 6 wherein the inflammation mediated disorder is pain.

20. The method of claim 6 wherein the inflammation mediated disorder is fever.

21. The method of claim 6 wherein the inflammation mediated disorder is a gastrointestinal disorder.

22. The method of claim 21 wherein the gastroinstestinal disorder is selected from the group consisting of inflammatory bowel disease, Crohn's disease, gastritis, irritable bowel syndrome and ulcerative colitis.

23. The method of claim 6 wherein the subject is a mammal.

24. The method of claim 23 wherein the mammal is a human.

25. The method of claim 6 wherein the cyclooxygenase-2 selective inhibitor or a pharmaceutically acceptable salt or prodrug thereof and acetaminophen or a pharmaceutically acceptable salt or prodrug thereof are administered in a sequential manner.

26. The method of claim 6 wherein the cyclooxygenase-2 selective inhibitor or a pharmaceutically acceptable salt or prodrug thereof and acetaminophen or a pharmaceutically acceptable salt or prodrug thereof are administered in a substantially simultaneous manner.

27. The method of claim 6 wherein the cyclooxygenase-2 selective inhibitor or a pharmaceutically acceptable salt or prodrug thereof and acetaminophen or a pharmaceutically acceptable salt or prodrug thereof are administered in a single dose form.

28. The method of claim 27 wherein the single dose form comprises about 0.1 to about 2000 milligrams of selective cyclooxygenase-2 inhibitor or a pharmaceutically acceptable salt or prodrug thereof.

29. The method of claim 27 wherein the single dose form comprises about 0.5 to about 500 milligrams of selective cyclooxygenase-2 inhibitor or a pharmaceutically acceptable salt or prodrug thereof.

30. The method of claim 27 wherein the single dose form comprises about 1 to about 200 milligrams of selective cyclooxygenase-2 inhibitor or a pharmaceutically acceptable salt or prodrug thereof.

31. The method of claim 30 wherein the single dose form comprises about 1000 to about 4000 milligrams of acetaminophen or a pharmaceutically acceptable salt or prodrug thereof.
